(19) United States
(12) Patent Application Publication
(10) Pub. No.: US 2011/0313196 A1
(43) Pub. Date: Dec. 22, 2011

(54) NOVEL PROCESS

(76) Inventors: Stephan Bachmann, Allschwil (CH); Fritz Bliss, Hartheim (DE); Ralph Diodone, Breisach (DE); Stefan Hildbrand, Gelterkinden (CH); Michael Kammerer, Basel (CH); Christophe Pfleger, Mulhouse (FR); Reinhard Reents, Muenchenstein (CH); Michelangelo Scalone, Birsfelden (CH)

(21) Appl. No.: 13/153,651

(22) Filed: Jun. 6, 2011

(30) Foreign Application Priority Data
Jun. 18, 2010 (EP) 10166461.3

(51) Int. Cl. C07C 231/12; C07C 209/60

(52) U.S. Cl. 564/139; 564/161; 564/374

(57) ABSTRACT

The present invention relates to a process for the preparation of benzamide derivatives which are useful as intermediates in the preparation of pharmaceutically active compounds.

### PRIORITY TO RELATED APPLICATION(S)

This application claims the benefit of European Patent Application No. 10166461.3, filed, Jun. 18, 2010, which is hereby incorporated by reference in its entirety.

### FIELD OF THE INVENTION

The present invention relates to a process for the preparation of benzamide derivatives which are useful as intermediates in the preparation of pharmaceutically active compounds.

### DESCRIPTION OF THE INVENTION

In a first aspect, the present invention provides a process for the preparation of a benzamide derivative of formula (I):

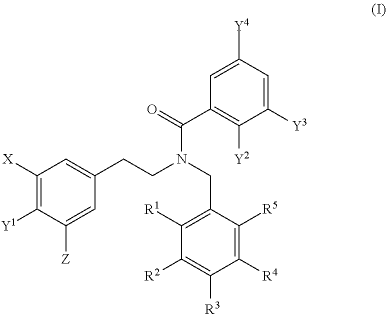

wherein
X and Z are independently hydrogen, halogen, (C₁-C₆)alkyl, halo(C₁-C₆)alkyl (in particular (C₁-C₃)perhaloalkyl), C₃-C₇cycloalkyl or halo(C₁-C₆)alkoxy;
Y¹, Y² and Y³ are independently hydrogen or halogen atoms;
Y⁴ is (C₁-C₆)alkyl or (C₁-C₃)perhaloalkyl;
R¹, R², R⁴ and R⁵ are independently hydrogen, halogen, (C₁-C₆)alkyl, (C₁-C₆)alkoxy;
R³ is halogen, (C₁-C₆)alkyl, hydroxy(C₁-C₆)alkyl, —Si((C₁-C₆)alkyl)₃ (C₁-C₆)alkoxy, halo(C₁-C₆)alkoxy, halo(C₁-C₆)alkyl, C₃-C₇cycloalkyl, phenyl, benzyl, —NR¹¹R¹², wherein R¹¹ and R¹² independently from each other are selected from hydrogen, C₁-C₆alkyl, and phenyl, or —C(O)—OR¹³, wherein R¹³ is hydrogen or C₁-C₆alkyl;
which comprises reacting compound of formula (II):

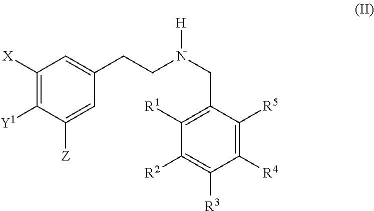

wherein X, Z, Y¹, R¹, R², R³, R⁴ and R⁵ are as defined above with a compound of formula (III):

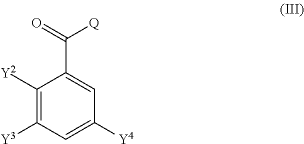

wherein Y², Y³ and Y⁴ are as defined above and Q is hydroxy or chloride in the presence of a tertiary amine with the proviso that when Q is hydroxy a coupling agent is present.

The compound of formula (III) is generally available from commercial sources or is readily prepared using methods well known to the person skilled in the art or prepared according to the procedures and examples described in WO2007/090748 and WO2009/147068.

In particular, X and Z are independently hydrogen, halogen, (C₁-C₆)alkyl, (C₁-C₃)perhaloalkyl, R¹, R², R⁴ and R⁵ are hydrogen and R³ is (C₁-C₆)alkyl.

In another embodiment, the present invention further provides a process for the preparation of compound of formula (III) wherein Q is chloride which comprises contacting compound of formula (III) wherein Q is hydroxy with oxalyl chloride or thionyl chloride.

The amine coupling, wherein Q is hydroxy may be performed in the presence of a solvent, ether-like solvent (e.g. tetrahydrofuran, diisopropyl ether, t-butylmethyl ether, dibutyl ether, dimethyl acetal or dioxane), ester-like solvent (e.g. ethyl acetate), aromatic solvent (e.g. toluene or t-butyl-benzene), an aliphatic hydrocarbon solvent (e.g. hexanes, heptanes or pentane), a saturated alicyclic hydrocarbon solvent (e.g. cyclohexane or cyclopentane), acetonitrile, an aprotic polar solvent (e.g. dimethylformamide), or dimethyl sulfoxide, in particular in the presence of tetrahydrofuran, toluene or dimethylformamide, more preferred in the presence of THF or toluene. The temperature may vary from 0-100° C., preferred from 15-50° C., most preferred at 20-25° C.

The acid chloride formation and the amine coupling wherein Q is chloride may particularly be preformed in the same solvent. The solvent may be selected from ether-like solvent (e.g. tetrahydrofuran, diisopropyl ether, t-butylmethyl ether, dibutyl ether, dimethyl acetal or dioxane), ester-like solvent (e.g. ethyl acetate), aromatic solvent (e.g. toluene or t-butyl-benzene), an aliphatic hydrocarbon solvent (e.g. hexanes, heptanes or pentane), a saturated alicyclic hydrocarbon solvent (e.g. cyclohexane or cyclopentane), acetonitrile, an aprotic polar solvent (e.g. dimethylformamide), or dimethyl sulfoxide, particularly in the presence of tetrahydrofuran, toluene or dimethylformamide, more preferred in the presence of toluene. In particular, the acid chloride formation is carried out in the presence of an N-Formamide such as DMF, N-Formylpiperidine or N,N-diphenyl formamide, more preferably in the presence of N,N-diphenyl formamide and DMF. The temperature may vary from 0-100° C., preferred from 15-50° C., most preferred at 20-25° C.

In the amine coupling wherein Q is hydroxy, the preferred coupling agent is EDC, EDC HCl, DCC, HBTU, TBTU, HOBT.

In particular the amine coupling is followed by a crystallisation.

The crystallization of compounds of formula (I) may be performed in the presence of a polar solvent such as alcohol (e.g. methanol, ethanol, 2-propanol, n-propanol), acetonitrile and water or combinations thereof. The API concentration before H₂O addition may vary from 10-50 w %, preferred from 15-40 w %, most preferred 20-30 w % as alcoholic or acetonitrile solution. The amount of water added may range from 0-50 w %, preferred 10-30 w %, most preferred 20 w %. The amount of seeding crystals added may vary from 0.1-10 w %, preferred 1-8 w %, most preferred 2-5 w %. The temperature whereupon the seeding crystals are added may vary from 0-40° C., preferred 5-30° C., most preferred 9-15° C. The crystallization temperature may range from 10 to (−)20° C., preferred 5 to (−)10° C., most preferred 0 to (−)5° C.

In another embodiment, the present invention provides a process for the preparation of a benzamide derivative of formula (I):

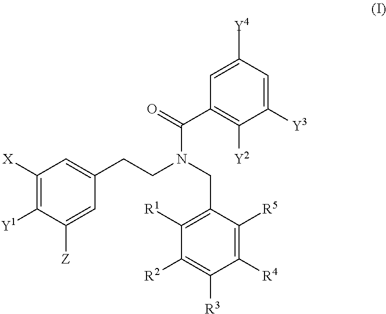

wherein
X and Z are independently hydrogen, halogen, (C₁-C₆)alkyl, halo(C₁-C₆)alkyl (in particular (C₁-C₃)perhaloalkyl), C₃-C₇cycloalkyl or halo(C₁-C₆)alkoxy;
Y¹, Y² and Y³ are independently hydrogen or halogen atoms;
Y⁴ is (C₁-C₆)alkyl or (C₁-C₃)perhaloalkyl;
R¹, R², R⁴ and R⁵ are independently hydrogen, halogen, (C₁-C₆)alkyl, (C₁-C₆)alkoxy;
R³ is halogen, (C₁-C₆)alkyl, hydroxy(C₁-C₆)alkyl, —Si((C₁-C₆)alkyl)₃ (C₁-C₆)alkoxy, halo(C₁-C₆)alkoxy, halo(C₁-C₆)alkyl, C₃-C₇cycloalkyl, phenyl, benzyl, —NR¹¹R¹², wherein R¹¹ and R¹² independently from each other are selected from hydrogen, C₁-C₆alkyl, and phenyl, or —C(O)—OR¹³, wherein R¹³ is hydrogen or C₁-C₆alkyl;
which comprises reacting compound of formula (II):

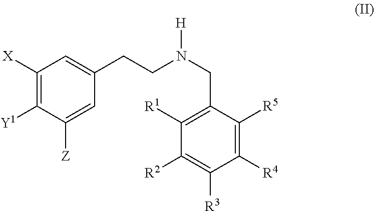

wherein X, Z, Y¹, R¹, R², R³, R⁴ and R⁵ are as defined above with a compound of formula (III):

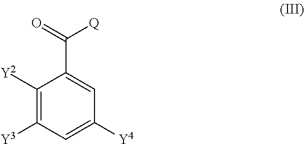

wherein Y², Y³ and Y⁴ are as defined above and Q is hydroxy in the presence of a tertiary amine and a coupling agent.

In a further embodiment, the present invention provides a process for the preparation of a benzamide derivative of formula (I):

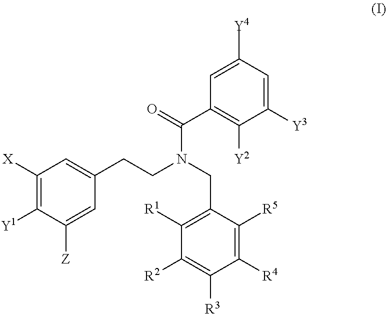

wherein
X and Z are independently hydrogen, halogen, (C₁-C₆)alkyl, halo(C₁-C₆)alkyl (in particular (C₁-C₃)perhaloalkyl), C₃-C₇cycloalkyl or halo(C₁-C₆)alkoxy;
Y¹, Y² and Y³ are independently hydrogen or halogen atoms;
Y⁴ is (C₁-C₆)alkyl or (C₁-C₃)perhaloalkyl;
R¹, R², R⁴ and R⁵ are independently hydrogen, halogen, (C₁-C₆)alkyl, (C₁-C₆)alkoxy;
R³ is halogen, (C₁-C₆)alkyl, hydroxy(C₁-C₆)alkyl, —Si((C₁-C₆)alkyl)₃ (C₁-C₆)alkoxy, halo(C₁-C₆)alkoxy, halo(C₁-C₆)alkyl, C₃-C₇cycloalkyl, phenyl, benzyl, —NR¹¹R¹², wherein R¹¹ and R¹² independently from each other are selected from hydrogen, C₁-C₆alkyl, and phenyl, or —C(O)—OR¹³, wherein R¹³ is hydrogen or C₁-C₆alkyl;
which comprises reacting compound of formula (II):

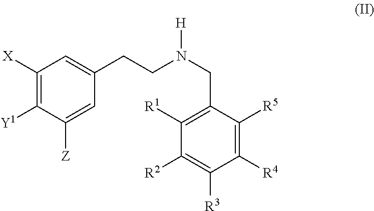

wherein X, Z, Y¹, R¹, R², R³, R⁴ and R⁵ are as defined above with a compound of formula (III):

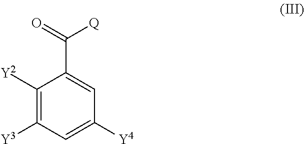

wherein Y², Y³ and Y⁴ are as defined above and Q is hydroxy in the presence of a tertiary amine and oxalyl chloride or thionyl chloride.

In another embodiment, the present invention provides a process for the preparation of compound of formula (II):

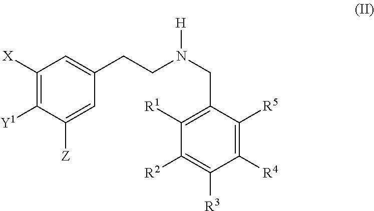

wherein X, Z, Y¹, R¹, R², R³, R⁴ and R⁵ are as defined above, which comprises the following steps:
a) reacting a compound of formula (IV):

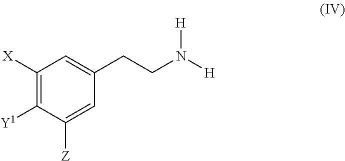

wherein X, Z and Y¹, are as defined above and, with a compound of formula (V):

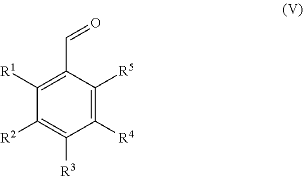

wherein R¹, R², R³, R⁴ and R⁵ are as defined above to obtain an imine derivative of formula (VI),

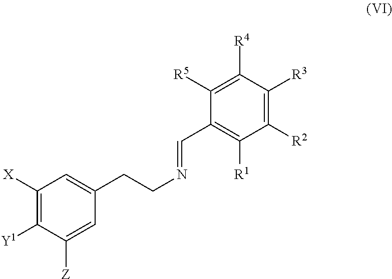

wherein X, Z, Y¹, R¹, R², R³, R⁴ and R⁵ are as defined above,
b) reacting the said imine derivative with H₂ in the presence of a metal catalyst, such as Pt/C, Pd/C, Ra—Ni, Ra—Co or a reducing agent such as NaBH₄, Na(OAc)₃BH, LiAlH₄, sodium bis(2-methoxyethoxy)aluminum hydride to obtain a compound of formula (II) with the proviso that when LiAlH₄ or Pd/C is used, none of the groups X, Z, Y¹, R¹, R², R³, R⁴ and R⁵ are chloride.
c) Should LiAlH₄ or Pd/C be used in accordance with step b) (the imine reduction), it is expected that the chloride would be partially cleaved from formula (VI).

Particularly in step b, the imine derivative of formula (VI) is reacted with a metal catalyst, most preferably with Pt/C, Ra—Ni or Ra—Co.

The compound of formula (IV) is synthesised according to procedures analogous to those described in T. Laue, A. Plagens “Namen- and Schlagwort-Reaktionen der Organischen Chemie”, Teubner Studienbücher, Stuttgart 1998 and references cited therein. For example to a compound of formula (VII) in the presence of an aqueous base and a solvent such as an ether-like solvent (e.g. tetrahydrofuran, 2-methyl-tetrahydrofuran, diisopropyl ether, t-butylmethyl ether, dibutyl ether, dimethyl acetal or dioxane), aromatic solvent (e.g. toluene or t-butyl-benzene), an aprotic polar solvents (e.g. dimethylformamide), ester-like solvent (e.g. ethyl acetate, 1-methyl-2-pyrrolidinone) and water, or mixture thereof, is added an oxidizing agent, such as a PhI(OAc)₂, PhI(OC(O)CF₃)₂, Pb(OAc)₄ or a hypohalite as defined above, as shown in Scheme 1 below. In particular, the oxidizing agent is PhI(OAc)₂. In Scheme 1, X, Y¹, Z are as defined above.

The compound of formula (I) and the herein described intermediates may be used as a valuable pharmaceutical compound or in the synthesis of valuable pharmaceutically compounds, such as the ones described in WO2007/090748.

Unless otherwise stated, the following terms used alone or as part of another group in the specification and claims have the meanings given below:

“(C₁-C₆)alkyl” refers to a branched or straight saturated hydrocarbon chain of one to six carbons atoms, such as methyl, ethyl, n-propyl, isopropyl, n-butyl, isobutyl, sec-butyl, t-butyl, pentyl and hexyl and their isomers thereof.

“(C₃-C₆)cycloalkyl” refers to a single saturated carbocyclic ring, such as cyclopropyl, cyclobutyl, cyclopentyl and cyclohexyl.

The term “halo” or “halogen” means fluorine, chlorine, bromine or iodine, in particular fluorine or chlorine.

The term “(C₁-C₃)perhaloalkyl” means an (C₁-C₃)alkyl group as defined above wherein all hydrogen atoms have been replaced with halogen atoms. More particularly “(C₁-C₃)perhaloalkyl” is (C₁-C₃)perfluoroalkyl, most preferably trifluoromethyl.

“Aqueous base” refers to a solution comprising a base and water. Numerous bases which readily dissolve in water are known in the art, such as alkali-hydroxide (e.g. sodium hydroxide, potassium hydroxide) or alkaline-hydroxide (e.g. magnesium hydroxide, calcium hydroxide). More preferably the aqueous base has a pH >12.

“(C₁-C₆)alkoxy” is understood as being an —O—(C₁-C₆)alkyl wherein (C₁-C₆)alkyl is as above defined, such as methoxy, ethoxy, isopropoxy.

“halo(C₁-C₆)alkyl” is understood as being an (C₁-C₆)alkyl chain as above defined substituted by one or more, same or different halogen atoms, such as 2-Br or 2-Cl-ethyl, 1-Cl-ethyl, 4-Cl-butyl, 2,2,2-trichloro-1,1-dimethylethyl.

“halo(C₁-C₆)alkoxy” is understood as being an (C₁-C₆)alkoxy as above defined substituted by one or more, same or different halogen atoms, such as 2-Br or 2-Cl-ethoxy, 1-Cl-ethoxy, 4-Cl-butoxy, 2,2,2-trichloro-1,1-dimethylethoxy.

“hydroxy(C₁-C₆)alkyl” is understood as being an (C₁-C₆)alkyl chain as above defined substituted by one or more hydroxy groups, such as methanol, ethanol, butanol or propanol.

“C₃₋₆ alkenyl” refers to a straight-chain or branched hydrocarbon residue comprising a carbon-carbon double bond, having three to six carbon atoms, provided that the carbon atom of the attachment point of the C₃₋₆ alkenyl to the rest of the molecule is not bonded to another carbon atom of the C₃₋₆ alkenyl by a carbon-carbon double bond. An example of a C₃₋₆ alkenyl is 2-propenyl.

Hypohalite refers to a hypohalous acid salt of formula MOX′ wherein X′ is an halogen atom and M is a alkali metal. Representative examples include, but are not limited to, NaOBr (i.e. from a solution of Br₂ and NaOH or NaOCl and NaBr solution), KOBr (i.e. from a solution of Br₂ and KOH or NaOCl and KBr).

“amine base” refers to an amine of formula (a)

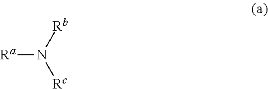

wherein R^{a}, R^{b} and R^{c} may be the same or different and are selected from the group consisting of hydrogen and (C₁-C₆)alkyl, or R^{a} is hydrogen or (C₁-C₆)alkyl and R^{b} and R^{c} taken together with the nitrogen atom to which they are attached, form a (C₄-C₈)heterocycloalkane optionally containing an additional heteroatom selected from O or N, with the proviso that at least one of R^{a}, R^{b} and R^{c} is not hydrogen. Representative examples include, but are not limited to dimethylamine, trimethylamine, diethylamine, diisopropylamine, ethyldiisopropylamine, ethylmethylamine, ethylpropylamine, methylpropylamine, N-Methyl morpholine, piperidine and tripropylamine. For the Heck coupling the preferred amine base is tripropylamine or ethyldiisopropylamine.

“tertiary amine” refers to an amine of formula (b)

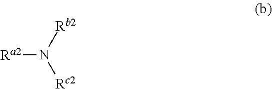

wherein R^{a2}, R^{b2} and R^{C2} may be the same or different and are (C₁-C₆)alkyl, cycloalkyl, phenyl or tolyl, or R^{a2} is (C₁-C₆)alkyl, R^{b2} and R^{c2} taken together with the nitrogen atom to which they are attached, form a (C₄-C₈)heterocycloalkane optionally containing an additional heteroatom selected from O or N. Representative examples include, but are not limited to trimethylamine, triethylamine, tributylamine, ethyldiisopropylamine, methylpropylamine, t-butyldimethylamine, diisopropylmethylamine, N-methyl morpholine, dimethylcyclohexylamine, dimethyl-toluidine, dimethylbenzylamine, triphenylamine, dicyclohexylmethylamine and tripropylamine.

For the amine coupling and the reductive amination the preferred tertiary amine is triethylamine.

“(C₄-C₈)heterocycloalkane” refers to a saturated non-aromatic cyclic compound of 4 to 8 ring atoms in which one or two ring atoms are heteroatoms selected from N or O, and the heterocycloalkane may be optionally substituted with one or more (C₁-C₃)alkyl, preferably one (C₁-C₃)alkyl.

“Inorganic base” refers to an alkali base, such as alkali carbonate, alkali bicarbonate, alkali borate, alkali phosphate or alkali-hydroxide.

### Synthetic Methods

The present invention provides novel methods for the synthesis of benzamides of formula I or intermediates therefor. Compounds of this invention can be synthesized according to the following general schemes. Suitable processes for synthesizing these compounds are provided in the examples.

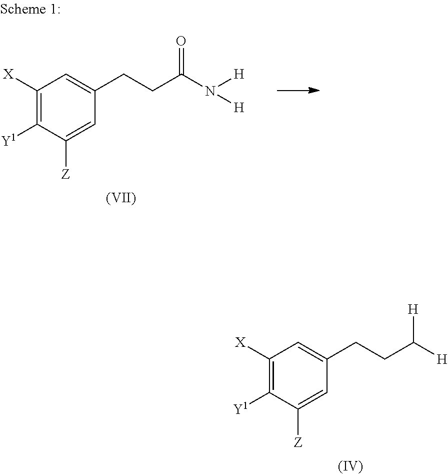

In a further embodiment, the present invention provides a process for the preparation of compound of formula (VII):

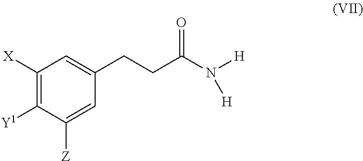

wherein X, Z, and Y¹ and are as defined above, which comprises reacting compound of formula (VIII):

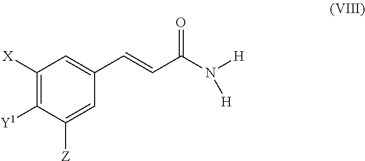

wherein X, Z, and Y¹ and are as defined above, with H₂ in the presence of a catalyst such as a Raney catalyst (e.g. Ra—Ni, Ra—Co) Pd/C or Pt/C, Pd/Alox, platinum oxide. More preferably the catalyst is a Raney catalyst, such as Ra—Ni or Ra—Co, or Pt/C.

In particular, for the hydrogenation of a compound of formula (VIII), the catalyst loading is between 1 and 30 w %, most preferably between 10-20 w %.

Preferably the hydrogenation is performed at temperatures between 20 and 100° C., more preferably between 20 and 50° C., most preferably 20 and 30° C.

The hydrogenation may be performed in the presence of a solvent, such as an alcohol (e.g. methanol or ethanol), an ether-like solvent (e.g. tetrahydrofuran, diisopropyl ether, t-butylmethyl ether, dibutyl ether, dimethyl acetal or dioxane), ester-like solvent (e.g. ethyl acetate), aromatic solvent (e.g. toluene or t-butyl-benzene), an aliphatic hydrocarbon solvent (e.g. hexanes, heptanes or pentane), a saturated alicyclic hydrocarbon solvent (e.g. cyclohexane or cyclopentane), an aprotic polar solvents (e.g. dimethylformamide), or dimethyl sulfoxide, preferably in the presence of methanol, ethanol, iso-propanol, tert-butanol, tetrahydrofuran, toluene or dimethylformamide, more preferred in the presence of ethanol or tetrahydrofuran.

The compound of formula (V) is generally available from commercial sources or is prepared according to the procedures and examples described in WO2007/090748.

The compound of formula (VIII) is synthesised according to procedures analogous to those described in J. Tsuji, “Transition Metal Reagents and Catalysts, Innovations in Organic Synthesis”, John Wiley & sons, Chichester, 2000 and references cited therein or T. Laue, A. Plagens “Namen- and Schlagwort-Reaktionen der Organischen Chemie”, Teubner Studienbücher, Stuttgart 1998 and references cited therein. For example to a compound of formula (IX) in presence of an amine base and a compound of formula (X) is added a palladium catalyst such as Pd(OAc)₂, PdCl₂, PdCl₂(PPh₃)₂, Pd(P(tBu)₃)₂, PdCl₂(BPPE) (BPPE: 1,2-bis(phenylphosphino)ethane) or Pd EnCat BINAP30® (encapsulated Pd-BINAP catalyst, commercially available from Reaxa/Sigma-Aldrich-Fluka®) or a combination of such a palladium complex either with a phosphine ligand such as PPh₃ or P(o-Tol)₃ or with an inorganic base such as NaOAc as defined above, as shown in Scheme 2 below. In particular, the catalyst is chosen from PdCl₂(PPh₃)₂, Pd(P(tBu)₃)₂, PdCl₂(BPPE) or Pd(OAc)₂/P(o-Tol)₃, most preferably the catalyst is Pd(OAc)₂/P(o-Tol)₃ or PdCl₂(PPh₃)₂. This step is in particular carried out in the presence of an organic solvent such as an ether-like solvent (e.g. tetrahydrofuran, diisopropyl ether, t-butylmethyl ether, dibutyl ether, dimethyl acetal or dioxane), an aprotic polar solvent (e.g. dimethylformamide) or a tertiary amine with a boiling point higher than 120° C. (e.g. tripropyl amine, diethylpropyl amine, diisopropylethylamine, N-methylmorpholine, N-methyl pyrrolidone). In Scheme 2, X, Z and Y¹ are as defined above and A is an halogen atom, toluenesulfonate or trifluoromethanesulfonate.

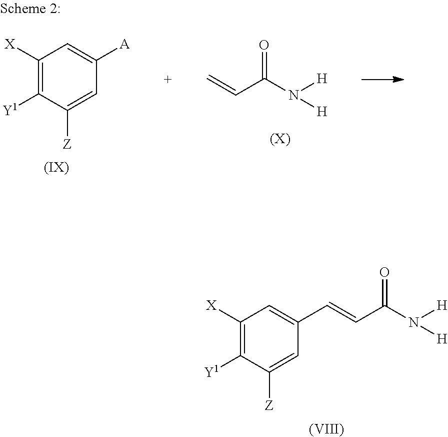

In another embodiment, the present invention provides a process for the preparation of the compound of formula (IV), which comprises the following steps:

1) the hydrogenation of compound of formula (VIII), as previously described;
2) the Hofmann degradation of compound of formula (VII), as previously described; preferably steps 1 and 2 are carried out as a one-pot synthesis, wherein X, Z and Y¹ are as defined above and the process is carried out according to Scheme 1′.

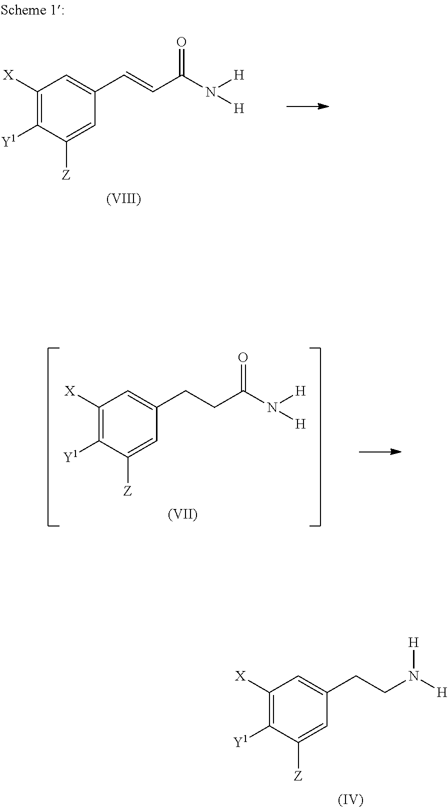

Most preferably the one-pot-synthesis process according to Scheme 1′ wherein compound of formula VII is not isolated, is carried out in tetrahydrofuran as the solvent with the exemplified reagents.

In a further embodiment the present invention provides a process for the preparation of the compound of formula (I), which comprises the following steps:

1) the reductive amination of compound of formula (IV) with compound of formula (V) to compound of formula (II) via compound of formula (VI), as previously described;
2) the amine coupling of compound of formula (II) with compound of formula (III), as previously described;
wherein Q, X, Z, Y¹, Y², Y³, Y⁴, R¹, R², R³, R⁴ and R⁵ are as defined above and the process is carried out according to Scheme 3.

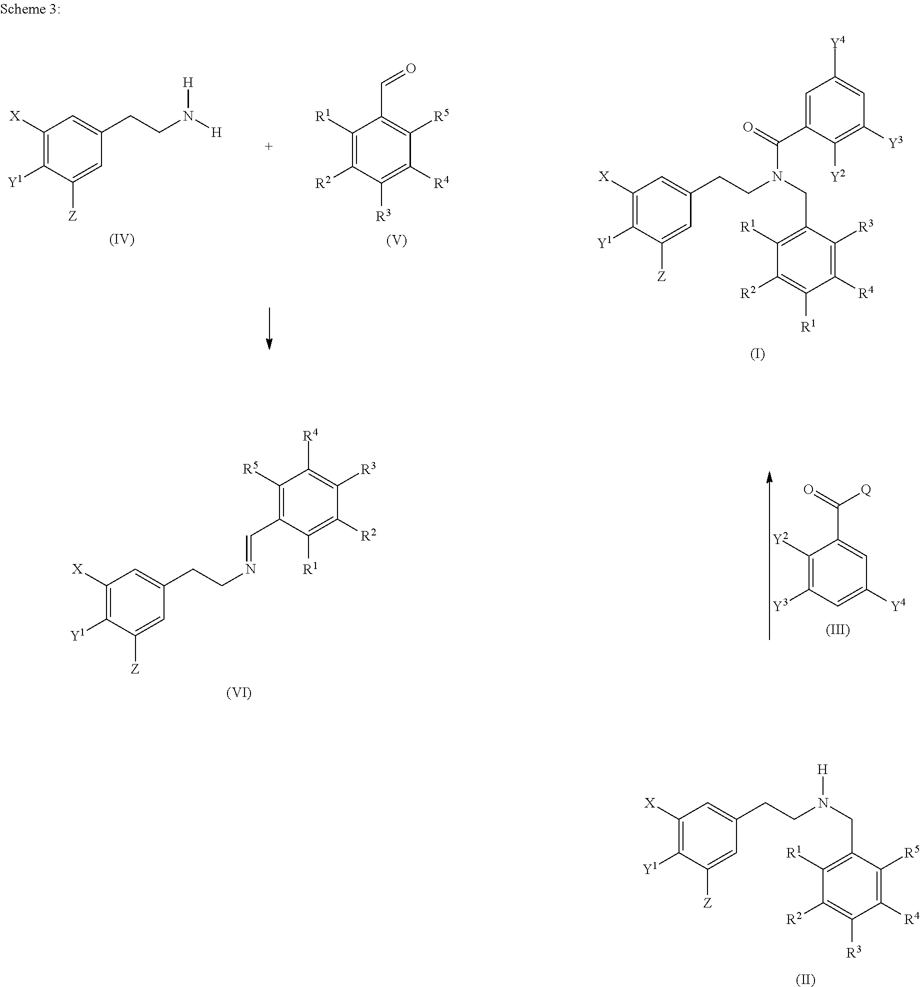

In a further embodiment the present invention provides a process for the preparation of the compound of formula (I), which comprises the following steps:

1) the Hofmann degradation of compound of formula (VII), as previously described;
2) the reductive amination of compound of formula (IV) with compound of formula (V) to compound of formula (II) via compound of formula (VI), as previously described;
3) the amine coupling of compound of formula (II) with compound of formula (III), as previously described;
wherein Q, X, Z, Y¹, Y², Y³, Y⁴, R¹, R², R³, R⁴ and R⁵ are as defined above and the process is carried out according to Scheme 4.

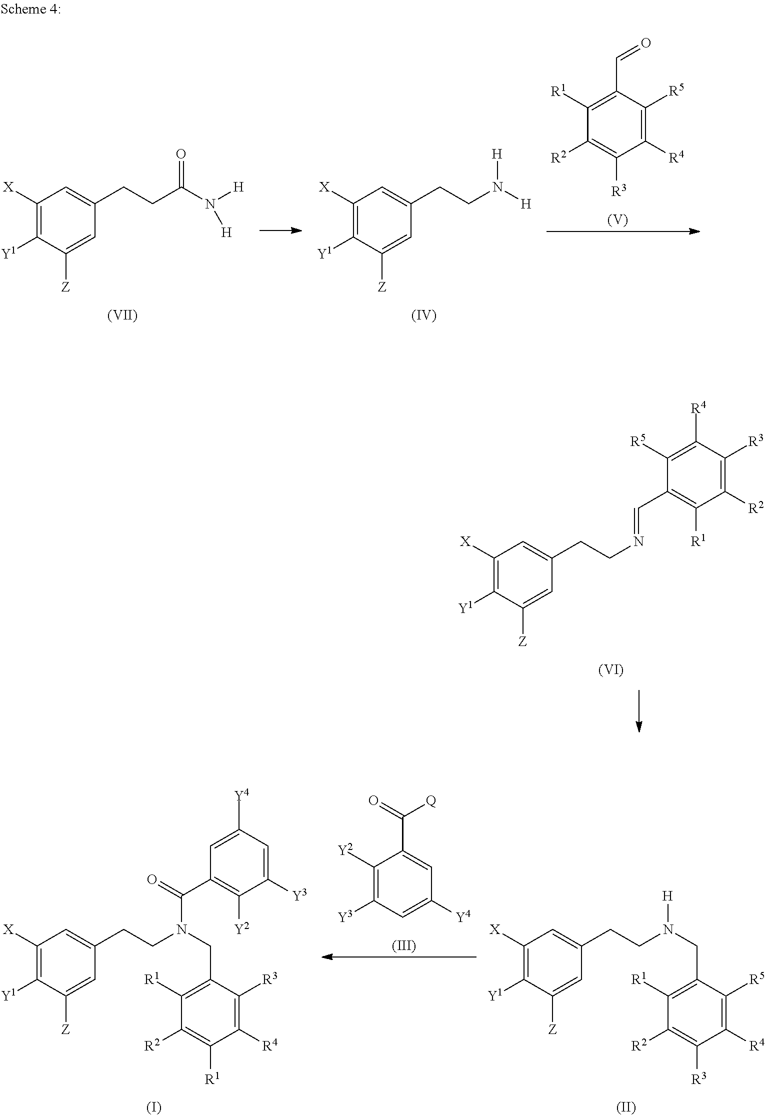

In a further embodiment the present invention provides a process for the preparation of the compound of formula (I), which comprises the following steps:

1) the hydrogenation of compound of formula (VIII), as previously described;
2) the Hofmann degradation of compound of formula (VII), as previously described;
3) the reductive amination of compound of formula (IV) with compound of formula (V) to compound of formula (II) via compound of formula (VI), as previously described;
4) the amine coupling of compound of formula (II) with compound of formula (III), as previously described;
wherein Q, X, Z, Y¹, Y², Y³, Y⁴, R¹, R², R³, R⁴ and R⁵ are as defined above and the process is carried out according to Scheme 5.

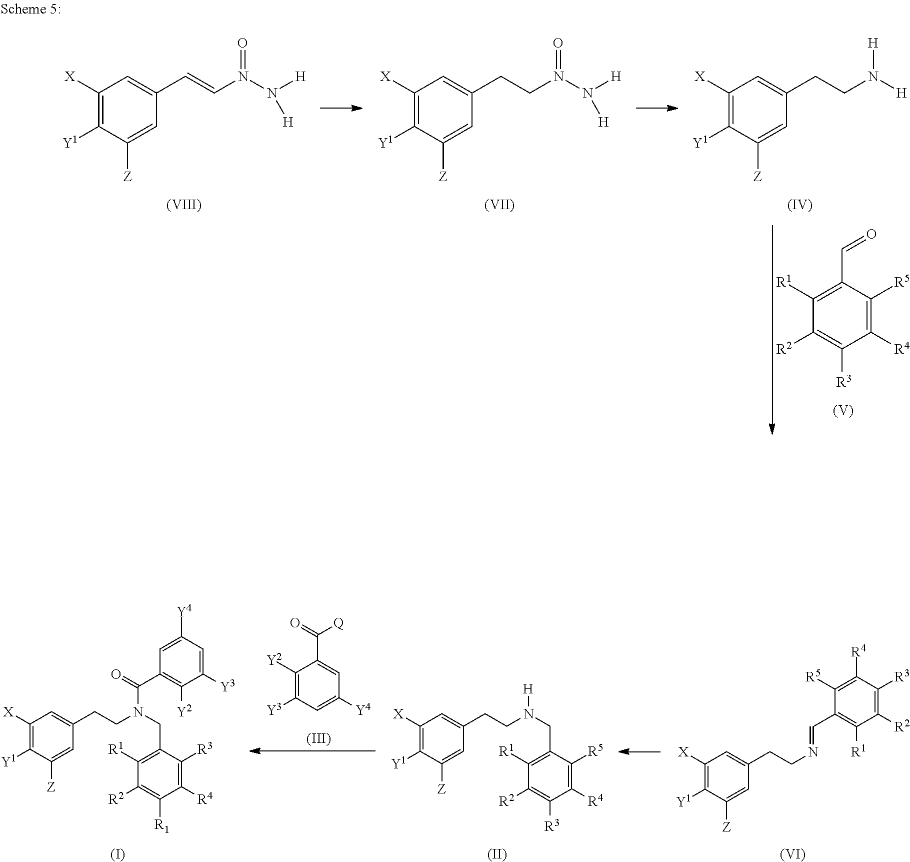

In a further embodiment the present invention provides a process for the preparation of the compound of formula (I), which comprises the following steps:

1) the Heck coupling of compound of formula (IX) with a compound of formula (X);
2) the hydrogenation of compound of formula (VIII), as previously described;
3) the Hofmann degradation of compound of formula (VII), as previously described;
4) the reductive amination of compound of formula (IV) with compound of formula (V) to compound of formula (II) via compound of formula (VI), as previously described;
5) the amine coupling of compound of formula (II) with compound of formula (III), as previously described;
wherein Q, X, Z, Y¹, Y², Y³, Y⁴, R¹, R², R³, R⁴ and R⁵ are as defined above and the process is carried out according to Scheme 6.

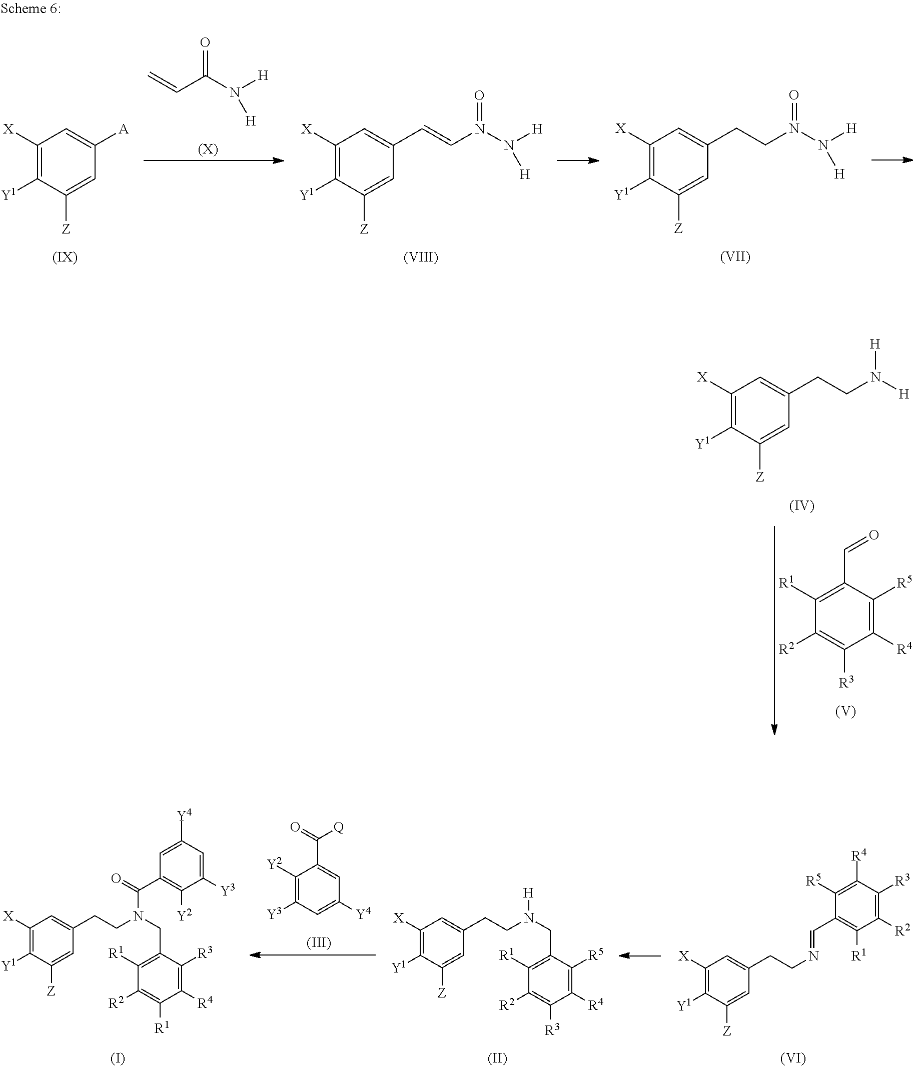

With exception of the hydrogenations of compound (VIII) to compound (VII) and compound (VI) to compound (II), all steps are performed preferably under an inert gas atmosphere, more preferably under argon or nitrogen.

The starting materials and reagents, which do not have their synthetic route explicitly disclosed herein, are generally available from commercial sources or are readily prepared using methods well known to the person skilled in the art.

The following examples are provided for the purpose of further illustration and are not intended to limit the scope of the claimed invention.

The following abbreviations and definitions are used: AcOEt (ethyl acetate); aq. (aqueous); Ar (argon); br (broad); BuLi (butyllithium); calcd. (calculated); CDCl₃ (deuterated chloroform); DCC(N,N′-dicyclohexylcarbodiimide); DCM (dichloromethane); DMF (dimethylformamide); EA (elemental analysis); EDC (1-Ethyl-3-(3-Dimethylaminopropyl)carbodiimide); EDC HCl (1-Ethyl-3-(3-Dimethylaminopropyl)carbodiimide hydrochloride); EI (electron impact); eq. (equivalent); EtOH (ethanol); g (gram); GC (gas chromatography); h (hour); H₂ (hydrogen); HBTU (O-Benzotriazole-N,N,N′,N′-tetramethyl-uronium-hexafluoro-phosphate); HCl (hydrochloric acid); HOBT (Hydroxybenzotriazole); HPLC (High-Performance Liquid Chromatography); ISP (isotopic Spin Population); KOH (Potassium Hydroxide); LDA (Lithium Diisopropylamide); LiAlH₄ (Lithium aluminium hydride); M (Molar); m (multiplet); m.p. (melting point); MS (Mass Spectroscopy); MeOH (methanol); mL (milliliter); m/z (mass per charge); N₂ (Nitrogen); NaBH₄ (sodium borohydride); NaCl (sodium chloride); NaHCO₃ (sodium bicarbonate); NaOMe (sodium methylate); NaOAc (sodium acetate); Na(OAc)₃BH (sodium triacetoxyborohydride); NaOH (sodium hydroxide); Na₂SO₄ (sodium sulfate); NEt₃ (triethylamine); NMP (1-methyl-2-pyrrolidone); NMR (nuclear magnetic resonance); Pb(OAc)₄ (lead acetate); Pd (palladium); Pd/C (palladium on Carbon); PdCl₂ (palladium chloride); Pd(OAc)₂ (palladium(II) acetate); PdCl₂(PPh₃)₂ (bis(triphenylphosphine)palladiumdichloride); Pd(P(tBu)₃)₂ (Bis(tri-tert-butylphosphine)palladium; PhI(OAc)₂ (Iodosobenzene Diacetate); PhI(OC(O)CF₃)₂ (bis(trifluoroacetoxy)iodobenzene); P(o-Tol)₃ (tris-(o-tolyl)-phosphine); PPh₃ (triphenylphosphine); PtO₂ (platinum oxide); Pt/C (platinum on carbon); Ra—Ni (Raney-Nickel); Ra—Co (Raney-Cobalt); Red-Al (sodium bis(2-methoxyethoxy)aluminum hydride); rpm (revolutions per minute); RT (room temperature); q (quartet); s (singlet); sat. (saturated); S/C (substrate/catalyst ratio); t (triplet); T3P (propanephosphonic acid anhydride); TBTU (o-Benzotriazol-1-yl-N,N,N′,N′-tetramethyluronium tetrafluoroborate); TBME (t-butyl methyl ether); THF (tetrahydrofuran); w/w (weight to weight).

### Example 1.1

### Synthesis of (E)-3-(4-Chloro-3-ethyl-phenyl)-acrylamide

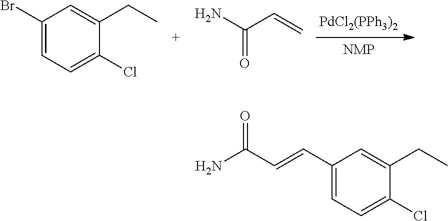

To a solution of 4-bromo-1-chloro-2-ethyl benzene (50 g, 227.8 mmol) and acrylic amide (19.4 g, 273.5 mmol, 1.2 eq.) in NMP (200 ml) was added under stirring and under Ar N-ethyldiisopropyl amine (47.8 ml, 273.5 mmol, 1.2 eq.) and PdCl₂(PPh₃)₂ (239.8 mg, 0.342 mmol, 0.0015 eq., S/C=650). The clear yellow solution was heated to 80° C. for 1 h, then the reaction temperature was increased to 100° C. and the solution was stirred at this temperature for additional 20 h. The brown reaction mixture was cooled to RT, filtered through paper and the filter cake was washed with MeOH (200 ml). After charcoal treatment and precipitation from MeOH/H₂O the title compound was obtained (44.0 g, 90.0% of theory) as light yellow crystals (m.p.: 152.4-153.2° C.). ¹H-NMR (400 MHz, CDCl₃): δ 7.59 (d, 1H), 7.38-7.27 (m, 3H), 6.44 (d, 1H), 5.85-5.55 (br d, 2H), 2.76 (q, 2H), 1.24 (t, 3H). MS (EI): m/z=208 ([M−H]⁺, 100%). EA for C₁₁H₁₂ClNO: calcd. C, 63.01; H, 5.77; N, 6.68. Found C, 62.75; H, 5.63; N, 6.50.

### Example 1.2

### Synthesis of (E)-3-(4-Chloro-3-ethyl-phenyl)-acrylamide

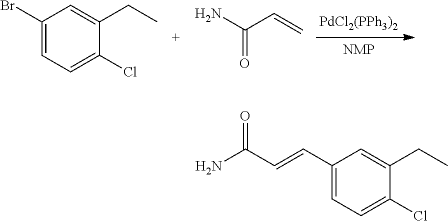

To a solution of 4-bromo-1-chloro-2-ethyl benzene (20 g, 91.12 mmol) and acrylic amide (7.77 g, 109.4 mmol, 1.2 eq.) in NMP (80 ml) was added under stirring and under Ar N-ethyldiisopropyl amine (19.1 ml) and PdCl₂(PPh₃)₂ (217.5 mg, 0.311 mmol, 0.0034 eq., S/C=300) was added. The clear yellow solution was heated to 80° C. for 1 h, then the reaction temperature was increased to 100° C. and stirred at this temperature for additional 20 h. The brown reaction mixture was cooled to RT, filtered through paper and the filter cake was washed with MeOH (200 ml). After charcoal treatment and precipitation from MeOH/H₂O the crude product (20.1 g, 105.2% of theory) was obtained as light yellow crystals. The crude product was re-crystallized from toluene/n-heptane (2:1) to yield the title compound (16.2 g, 83.5% of theory) as light yellow crystals.

### Example 1.3

### Synthesis of (E)-3-(4-Chloro-3-ethyl-phenyl)-acrylamide

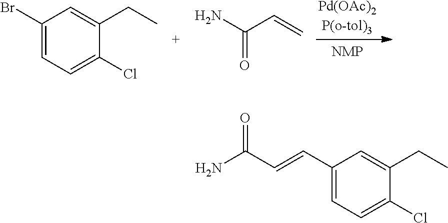

A clear yellow solution of acrylic amide (39.0 g, 548 mmol, 1.2 eq.), Pd(OAc)₂ (532 mg, 2.37 mmol, 0.0052 eq., S/C 200) and P(o-Tol)₃ (3.08 g, 10 mmol, 0.022 eq.) in DMF (143 ml) was treated sequentially with 4-bromo-1-chloro-2-ethyl benzene (100 g, 456 mmol), tripropyl amine (84.0 g, 575 mmol, 1.26 eq.) and DMF (143 ml). The reaction mixture was heated under vigorous stirring to 130° C. within 30 min, stirred at this temperature for 8 h and then cooled to 30° C. At this temperature, 2-methyltetrahydrofuran (620 ml) and NaCl solution (10%, 500 ml) were added and the mixture was stirred for 10 min. The reaction mixture was filtered, the filter cake was washed with 2-methyltetrahydrofuran (43 ml) and the phases were separated. The aq. phase was treated with 2-methyltetrahydrofuran (560 ml) and the phases were separated again. The combined organic phases were treated with HCl (1N, 500 ml) and the phases were separated. The clear, yellow organic phase was evaporated to give a yellow residue, which was taken up in 2-propanol (350 ml) at 60° C. The yellow suspension was stirred at 75° C. until a clear solution was obtained. The solution was concentrated under vacuum to a residual volume of 300 ml, the solution was cooled to 65° C. and seeding crystals (100 mg) were added. The mixture was stirred for 30 min until the crystallization has been initiated. The obtained suspension was cooled to 20° C. within 2 h and n-heptane (450 ml) was added over a period of 30 min. The light yellow suspension was cooled to −15° C. over a period of 2 h, the crystals were filtered off, washed with ice cold n-heptane/2-propanol (3:2, 125 ml) and the dried under vacuum at 50° C. until weight constancy to give the title compound as light yellow crystals (74.3 g, 77% of theory).

### Example 1.4

### Synthesis of (E)-3-(3-ethyl-phenyl)-acrylamide

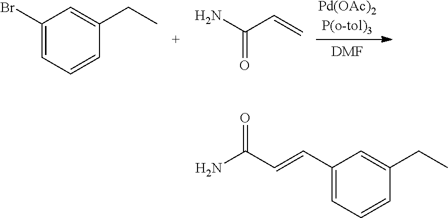

To a solution of Pd(OAc)₂ (59.4 mg 0.265 mmol, 0.005 eq., S/C=200) and P(o-Tol)₃ (179.1 mg, 0.583 mmol, 0.011 eq.) in DMF (10 ml) was added under stirring and under Ar sequentially a solution of 1-bromo-3-ethyl-benzene (10.0 g, 52.96 mmol) in DMF (20 ml) and a solution of acrylic amide (4.52 g, 63.55 mmol, 1.2 eq.) in DMF (20 ml). Afterwards N-ethyldiisopropyl amine (11.1 ml, 63.55 mmol, 1.2 eq.) was added and the reaction mixture was heated to 130° C. for 2 h. The reaction mixture was cooled to RT, filtered over dicalite, washed with DMF (120 ml) and the reaction mixture was concentrated to circa half of its volume. After charcoal treatment, filtration, extraction with AcOEt and removal of the solvent under vacuum, the title compound (8.90 g, 90.2% of theory) was obtained as light brown semi solid compound. ¹H-NMR (300 MHz, CDCl₃): δ 7.63 (d, 1H), 7.33-7.18 (m, 4H), 6.46 (d, 1H), 5.63 (br s, 2H), 2.67 (q, 2H), 1.25 (t, 3H). MS (EI): m/z=176 ([M+H]⁺, 100%).

### Example 1.5

### Synthesis of (E)-3-(3-Fluoro-3-trifluoromethyl-phenyl)-acrylamide

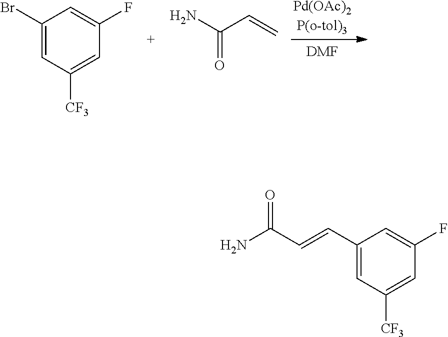

To a solution of Pd(OAc)₂ (46.2 mg 0.206 mmol, 0.005 eq., S/C=200) and P(o-Tol)₃ (139.2 mg, 0.453 mmol, 0.011 eq.) in DMF (10 ml) was added under stirring and under Ar sequentially a solution of 3-bromo-5-fluoro-benzotrifluoride (10.0 g, 41.15 mmol) in DMF (20 ml) and a solution of acrylic amide (3.51 g, 49.38 mmol, 1.2 eq.) in DMF (20 ml). Afterwards N-ethyldiisopropyl-amine (8.63 ml, 49.38 mmol, 1.2 eq.) was added and the reaction mixture was heated to 130° C. for 1.5 h. After charcoal treatment and precipitation from MeOH/H₂O the title compound (8.95 g, 91.1% of theory) was obtained as light green crystals (m.p.: 120.6-121.9° C.). ¹H-NMR (400 MHz, CDCl₃): δ 7.63 (m, 1H), 7.55 (s, 1H), 7.37 (d, 1H), 7.32 (s, 1H), 6.56 (d, 1H), 5.99 (br d, 2H). MS (EI): m/z=292 ([M+OAc]⁺, 100%). EA for C₁₀H₇F₄NO: calcd. C, 51.51; H, 3.03; N, 6.86. Found C, 51.51; H, 2.93; N, 6.92.

### Example 1.6

### Synthesis of (E)-3-(4-Chloro-3-ethyl-phenyl)-acrylamide

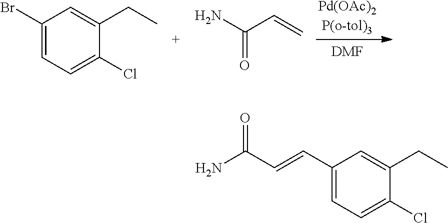

A 1000 L double-jacket reactor was charged with palladium(II)-acetate (122 mg, 0.55 mmol), tris-(o-tolyl)-phosphine (0.98 g, 2.46 mmol), acrylic amide (31.2 g, 430 mmol) and DMF (450 mL). 4-Bromo-1-chloroethylbenzene (80.0 g, 364 mmol) and tripropyl amine (67.1 g, 459 mmol) were then added and the resulting mixture was heated with a rate of 0.7° C./minute to 155° C. (jacket temperature) and then stirred at this temperature for additional 6 hours. The jacket temperature was set to 90-100° C. and approx. 370 g of DMF were distilled off under reduced pressure. The residue was cooled to 35° C. and treated with toluene (250 mL). The mixture was polish filtered and the filtrate poured into a hot mixture (70-75° C.) of water (390 mL) and conc. sulfuric acid (10 mL). The reactor, the pipes and the filter were rinsed with a mixture of toluene (150 mL) and DMF (10 mL). The resulting biphasic mixture was heated to 70° C. and the layers were allowed to separate. The aqueous layer was removed and back extracted at 70-75° C. with toluene (200 mL). The combined toluene layers were washed at 75-80° C. with water (1×100 mL) and then concentrated to a volume of 700-750 mL. Isopropanol (15 mL) was added at 90 to 95° C. and the resulting clear solution was allowed to cool to 10° C. within 5 to 10 hours, whereupon crystals precipitated. The suspension was stirred for 3 hours at 10° C. The crystals were filtered off, washed with toluene and dried at 50° C. and <30 mbars over night to yield the title compound as colorless crystals (62.8 g, 82% yield) with an assay of 99.5% w/w (determined by HPLC).

### Example 2.1

### Synthesis of 3-(4-Chloro-3-ethyl-phenyl)-propionamide

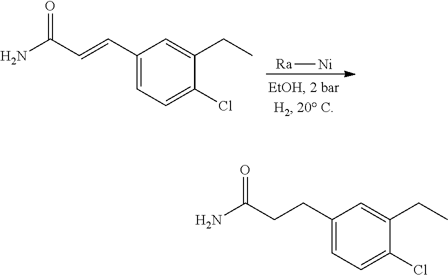

To a suspension of Ra—Ni (3.0 g, 25.55 mmol, 0.357 eq., 20% w/w) in EtOH (30 ml) was added under Ar in an autoclave a solution of (E)-3-(4-Chloro-3-ethyl-phenyl)-acrylamide (15.0 g, 71.54 mmol) in EtOH (40 ml). The autoclave was pressurized with 2 bar of H₂ and the reaction was run at 2 bar H₂ pressure at 20° C. for 75 min. The pressure was released from the autoclave, the suspension was filtered under Ar protection over dicalite and washed with THF. The filtrate was evaporated under reduced pressure and the resulting light yellow solid was dried under vacuum until weight constancy to yield the title compound (13.95 g, 92.1% of theory) as light yellow solid (m.p.: 122.8-123.3° C.). ¹H-NMR (400, CDCl₃): δ 7.24 (d, 1H), 7.07 (d, 1H), 6.97 (dd, 1H), 5.51 (br s, 1H), 5.36 (br s, 1H), 2.93 (m, 2H), 2.72 (q, 2H), 2.51 (m, 2H), 1.22 (t, 3H). MS (EI): m/z=212 ([M+H]⁺, 100%). EA for C₁₁H₁₄ClNO: calcd. C, 62.41; H, 6.67; N 6.62. Found C, 62.42; H, 6.37; N, 6.61.

### Example 2.2

### Synthesis of 3-(4-Chloro-3-ethyl-phenyl)-propionamide

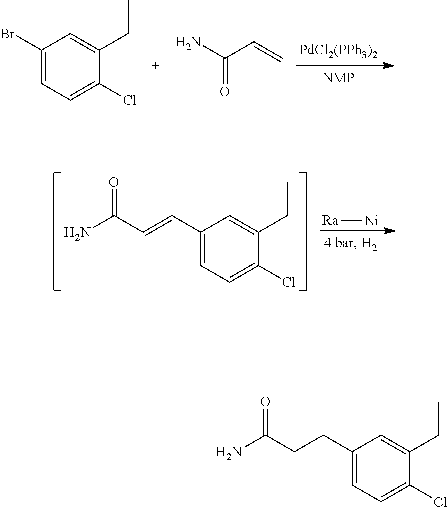

To a solution of Pd(OAc)₂ (11.2 mg, 0.05 mmol, 0.001 eq., S/C=1000) and NaOAc (4.97 g, 60.0 mmol, 1.2 eq.) in NMP (20 ml) was added under stirring and under Ar sequentially a solution of acrylic amide (4.26 g, 60.0 mmol, 1.2 eq.) in NMP (10 ml) and 4-bromo-1-chloro-2-ethyl-benzene (10.97 g, 50.0 mmol) in NMP (10 ml) and the reaction mixture was heated to 135° C. and stirred for 2 h at this temperature. Then the reaction temperature was increased to 155° C. and kept at this temperature for 4 h. The dark brown reaction mixture was cooled to RT and then filtered over dicalite, the filter cake was washed with AcOEt (50 ml), the filtrate was filtered again over paper and the filter cake was washed with additional AcOEt (5 ml). The clear dark brown filtrate was then directly loaded into an autoclave containing Ra—Ni (2.0 g, 17.38 mmol, 0.38 eq.) in AcOEt (4 ml). The autoclave was pressurized with 4 bar of H₂ and the reaction mixture was heated to 40° C. and run at 4 bar H₂ pressure for 5.5 h. The pressure was released from autoclave after cooling to RT, the suspension was filtered under Ar protection over dicalite and washed with AcOEt and water was added. The aq. phase was washed with AcOEt (250 ml), whereas the organic phase was washed with water (150 ml). The combined organic phases were concentrated under vacuum at 50° C. to a weight of 39 g and n-heptane (100 ml) was added and n-heptane was partially removed to give a thick suspension. This white suspension was stirred for 1 h at 50° C., 2 h at RT and 3 h in an ice bath. The crystals were filtered off, washed with n-heptane (30 ml) and dried under vacuum at 50° C. until weight constancy to yield the title compound (9.47 g, 87.8% of theory) as white crystals.

### Example 2.3

### Synthesis of 3-(3-ethyl-phenyl)-propionamide

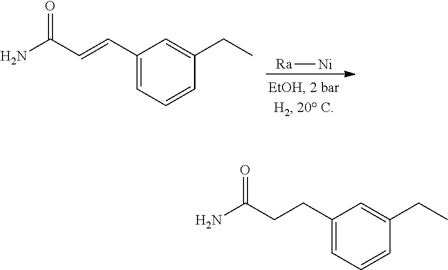

To a suspension of Ra—Ni (1.2 g, 10.22 mmol, 0.6 eq., 20% w/w) in EtOH (11 ml) was added under Ar in an autoclave a solution of (E)-3-(3-ethyl-phenyl)-acrylamide (6.0 g, 34.24 mmol) in EtOH (30 ml). The autoclave was pressurized with 2 bar of H₂ and the reaction was run at 2 bar H₂ pressure at 20° C. for 75 min. The pressure was released from the autoclave, the suspension was filtered under Ar protection over dicalite and washed with THF. The filtrate was evaporated under reduced pressure and the resulting light yellow solid was dried under vacuum until weight constancy to yield the title compound (5.35 g, 88.1% of theory) as light yellow solid. ¹H-NMR (400, CDCl₃): δ 7.21 (m, 1H), 7.04 (m, 2H), 5.64 (br s, 1H), 5.46 (br s, 1H), 2.95 (m, 2H), 2.63 (q, 2H), 2.53 (m, 2H), 1.22 (t, 3H). MS (EI): m/z=178 ([M+H]⁺, 100%).

### Example 2.4

### Synthesis of 3-(3-Fluoro-5-trifluoromethyl-phenyl)-propionamide

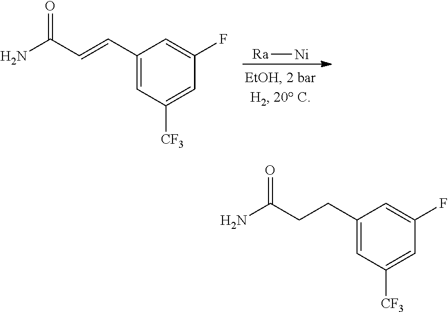

To a suspension of Ra—Ni (1.2 g, 10.15 mmol, 0.18 eq., 10% w/w) in EtOH (20 ml) was added under Ar in an autoclave a solution of (E)-3-(3-Fluoro-5-trifluoromethyl-phenyl)-acrylamide (13.0 g, 55.75 mmol) in EtOH (46 ml). The autoclave was sealed under an Ar flow, connected to the hydrogenation line and cooled to 20° C. The autoclave was pressurized 2 bar of hydrogen and the reaction was run at 2 bar H₂ pressure at 20° C. for 90 min. The pressure was released from the autoclave, the suspension was filtered under Ar protection over dicalite and washed with THF. The filtrate was evaporated under reduced pressure and the resulting yellow solid was dried under vacuum until weight constancy to yield the title compound (13.02 g, 94.3% of theory) as light yellow solid (m.p.: 76.9-77.8° C.). ¹H-NMR (400, CDCl₃): δ 7.27 (s, 1H), 7.18 (d, 1H), 7.13 (d, 1H), 5.88 (br s, 1H), 5.51 (br s, 1H), 3.03 (dd, 2H), 2.55 (dd, 2H). MS (EI): m/z=236 ([M+H]⁺, 100%). EA for C₁₀H₉F₄NO: calcd. C, 51.07; H, 3.86; N, 6.80. Found C, 51.31; H, 3.80; N, 6.78.

### Example 3.1

### Synthesis of 2-(4-Chloro-3-ethyl-phenyl)-ethylamine

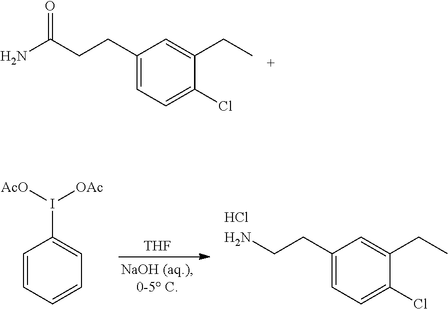

To a solution of 3-(4-Chloro-3-ethyl-phenyl)-propionamide (5.0 g, 23.62 mmol) in THF (45 ml) was added under stirring and under Ar in an ice bath (0-5° C.) NaOH (3 M, 50.1 ml, 150.2 mmol, 6.36 eq.) such that the temperature did not exceed 10° C. To this biphasic mixture was added slowly PhI(OAc)₂ (8.54 g, 25.98 mmol, 1.1 eq.) in small portions over a period of 40 min and the mixture was stirred for an additional hour. The reaction mixture was quenched and the pH of the reaction mixture was adjusted to ca. 1 upon addition of HCl (37%, 14.7 ml 173.1 mmol, 7.33 eq.). Toluene (30 ml) was added, the organic phase was separated and washed 5 times with HCl (2 M, 100 ml) whereas the aq. phases were washed with toluene (50 ml). To the combined aq. phases was added NaOH (32%, 20 ml, 216.0 mmol, 9.14 eq.), the pH was adjusted to 14 and toluene (50 ml) was added. The organic phase was separated, washed with water and brine, whereas the aq. phases were washed with toluene. The combined organic phases were dried over Na₂SO₄ and concentrated under vacuum to a volume of ca. 20 ml. The pH of this solution was adjusted to ca. 1 by addition of HCl in MeOH (2.5 M) (10 ml, 25.04 mmol, 1.06 eq.). From the resulting suspension the solvent was removed, AcOEt (30 ml) was added and the suspension was stirred for 1 h in an ice bath. The formed crystals were filtered off, washed with AcOEt and dried under vacuum at 50° C. until weight constancy to yield the title compound (4.39 g, 84.0% of theory) as white crystals (m.p.: 146.0-147.0° C.). ¹H-NMR (400 MHz, DMSO): δ 8.14 (br s, 3H), 7.36 (d, 1H), 7.25 (d, 1H), 7.12 (dd, 1H), 3.01 (m, 2H), 2.88 (m, 2H), 2.68 (q, 2H), 1.18 (t, 3H). MS (EI): m/z=184 ([M+H]⁺, 100%). EA for C₁₀H₁₅Cl₂N: calcd. C, 54.56; H, 6.87; N, 6.36. Found C, 54.32; H, 6.77; N, 6.60.

### Example 3.2

### Synthesis of 2-(4-Chloro-3-ethyl-phenyl)-ethylamine

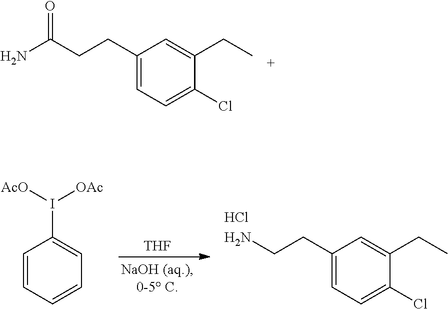

To a solution of 3-(4-Chloro-3-ethyl-phenyl)-propionamide (40.0 g, 187 mmol, 1.0 eq.) in THF (240 ml) was added at 0° C. within 20 min NaOH (3.4 M, 271 ml, 910 mmol, 4.9 eq.) whereupon the temperature increased to 20° C. The corresponding bi-phasic mixture was cooled to −3° C. and PhI(OAc)₂ (67.6 g, 210 mmol, 1.12 equiv) was added portionwise. After complete addition, the reaction mixture was stirred for 1 h followed by the addition of HCl (37%, 110 ml, 1.31 mol, 7.0 eq.). To the turbid bi-phasic mixture was added at 20° C. toluene (120 ml) and the mixture was stirred for 10 min. The phases were separated, the lower aq. phase was removed, the organic phase was treated with HCl (3.1 M, 85 ml, 264 mmol, 1.4 eq.) and the mixture was stirred for 10 min. The phases were separated, the combined aq. phases were treated at RT with NaOH (28%, 140 ml, 1.32 mmol, 7.0 eq.), toluene (160 ml) was added, the mixture was stirred for 10 min. The phases were separated, the organic phase was treated with water (320 ml) and the mixture was stirred for 10 min. The phases were separated, the organic phase was treated with HCl (37%, 24 ml, 282 mmol, 1.5 eq.), toluene (320 ml) was added and the water was removed upon azeotrope distillation with toluene adjusting the volume to 300 ml. The corresponding white suspension was cooled to −10° C. within 1 h, and the suspension was stirred at this temperature for 1 h. The crystals were filtered off, washed with toluene (100 ml) and dried under vacuum at 50° C. for 14 h to give the title compound as white crystals (35.9 g, 86% of theory).

### Example 3.3

### Synthesis of 2-(3-ethyl-phenyl)-ethylamine

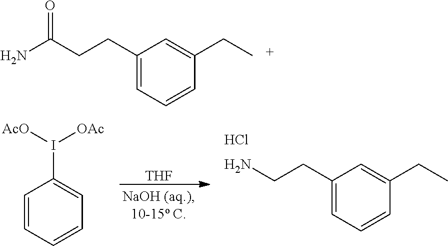

To a solution of 3-(3-ethyl-phenyl)-propionamide (4.0 g, 22.57 mmol) in THF (36 ml) was added under stirring and under Ar in an ice bath NaOH (3 M, 23.9 ml, 71.77 mmol, 3.18 eq.) and water (20 ml) such that the temperature did not exceed 10° C. To this biphasic mixture was added portionwise at 0-5° C. PhI(OAc)₂ (7.79 g, 23.70 mmol, 1.05 eq.). The mixture was stirred for an additional hour. The reaction mixture was quenched and the pH of the reaction mixture was adjusted to 1 upon addition of HCl (25%, 14.3 ml, 440.1 mmol, 19.5 eq.). The organic phase was separated and washed twice with water (50 ml) whereas the aq. phases were washed with AcOEt (50 ml). To the combined aq. phases was added NaOH (32%, 9.55 ml, 103.1 mmol, 4.57 eq., note 13), the pH was adjusted to 14 and AcOEt (50 ml) was added. The organic phase was separated, washed with water and brine, whereas the aq. phases were washed with AcOEt. The combined organic phases were dried over Na₂SO₄ and concentrated under vacuum to dryness. The residue was dissolved in AcOEt (30 ml) and the pH of the solution was adjusted to 1 by adding HCl (37%, 2.0 ml, 23.63 mmol, 1.05 eq.). From the resulting suspension the solvent was removed, AcOEt (30 ml) was added and the suspension was stirred for 1 h in an ice bath. The formed crystals were filtered off, washed with AcOEt and dried under vacuum at 50° C. until weight constancy to yield the title compound (2.46 g, 58.7% of theory) as white crystals. ¹H-NMR (400 MHz, DMSO): δ 8.17 (br s, 3H), 7.24 (m, 1H), 7.12-7.04 (m, 3H), 3.00 (m, 2H), 2.88 (m, 2H), 2.59 (q, 2H), 1.18 (t, 3H). MS (EI): m/z=150 ([M+H]⁺, 100%).

### Example 3.4

### Synthesis of 2-(3-Fluoro-5-trifluoromethyl-phenyl)-ethylamine

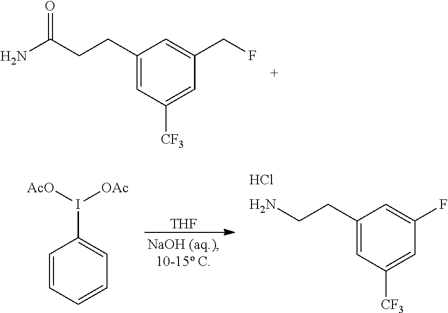

To a solution of 3-(3-Fluoro-5-trifluoromethyl-phenyl)-propionamide (5.0 g, 21.26 mmol) in THF (50 ml) was added under stirring and under Ar in an ice bath (10° C.) NaOH (2.78 g, 68.24 mmol, 3.21 eq.) in water (50 ml). To this biphasic mixture was added slowly at 10° C. PhI(OAc)₂ (7.69 g, 23.4 mmol, 1.1 eq.) portionwise over a period of 25 min. The mixture was stirred for 2 h and the THF was exchanged with TBME under vacuum. The phases were separated, the organic phase was washed with water (50 ml), whereas the aq. phase was washed with TBME (70 ml). The combined organic phases were dried over Na₂SO₄ and the solvent was removed under vacuum. The residue was taken up in n-heptane (50 ml) and HCl (25%, 8.2 ml) was added dropwise under cooling (ice bath) whereupon the pH was adjusted to 1 and a light yellow precipitate was formed. The suspension was stirred for 46 h at RT, the crystals were filtered off, washed with n-heptane and dried under vacuum at 50° C. until weight constancy to yield the title compound (4.82 g, 93.1% of theory) as light yellow crystals (m.p.: >170° C. decomposition). ¹H-NMR (400 MHz, DMSO): δ 8.14 (br s, 3H), 7.55 (m, 3H), 3.12 (m, 2H), 3.03 (m, 2H). MS (EI): m/z=208 ([M+H]⁺, 100%). EA for C₉H₁₀ClF₄N: calcd. C, 44.37; H, 4.14; N, 5.75. Found C, 44.13; H, 3.93; N, 5.75.

### Example 3.5

### Telescoped Process for the Synthesis of 2-(4-Chloro-3-Ethyl-Phenyl)-Ethyl-Amine-Hydrochloride

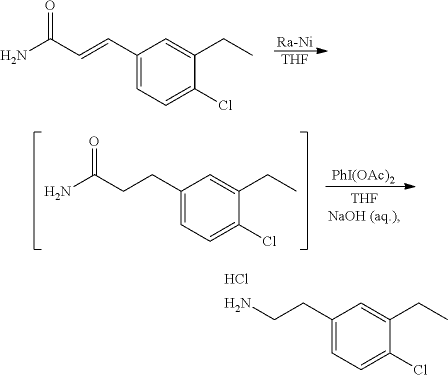

A suspension of wet Raney-Nickel (46.5%; 8.6 g, 0.07 mol) and (E)-3-(4-chloro-3-ethyl-phenyl)-acryl amide (80.0 g, 0.38 mol) in THF (320 g) was pressurized with 4 bar hydrogen and hydrogenated at 25 to 35° C. for 4 hours. Upon complete conversion (<0.2% starting material) the catalyst was filtered off and rinsed with THF (100 g). The filtrate was cooled to 0° C. and water (350 g) followed by sodium hydroxide (28% in water; 260 g) was added at a maximum temperature of 15° C. (Diacetoxy)-iodbenzene (133.0 g, 0.413 mol) was then added in 8 portions at −3 to 3° C. within 1.5 hours. Upon complete conversion the pH of the mixture was adjusted to pH 5.8-6.2 by addition of hydrochloric acid (37% in water; ca. 143 g). From the mixture approx. 350 mL of THF/water where distilled off. The pH of the remaining suspension was adjusted to pH 3-4 by addition of hydrochloric acid (37% in water; ca. 62 g). Toluene (210 g) was added and the layers were allowed to separate. From the resulting tri-phasic mixture the two lower layers were separated and the upper toluene layer was extracted with water (1×120 g). The combined aqueous (product-containing) layers were treated with potassium hydroxide (50% in water; ca. 126 g) until a pH of 11-12 was obtained. Toluene (275 g) was added and the layers were allowed to separate. The aqueous layer was removed and the organic layer washed at 35-40° C. with water (1×150 g). The organic layer was treated at 35-40° C. with hydrochloric acid (37% in water; 56.6 g). From the resulting mixture water/toluene was distilled off at 50 to 70° C. and the distillate was continuously replaced by toluene. In total 500 g of toluene were added. At the end of the distillation a volume of approx. 600 mL was adjusted in the reactor. The mixture was cooled from 70° C. to −10° C. within 4 hours, whereupon crystals precipitated. The resulting suspension was stirred for 3 hours at −10° C. The crystals were filtered off, washed with toluene (174 g) and dried at 50° C. and <30 mbars over night to yield the title compound as colorless crystals (78.5 g, 93% yield) with an assay of 99.8% w/w (determined by HPLC).

### Example 4.1

### Synthesis of (4-tert-butyl-benzyl)-2-(4-chloro-3-ethyl-phenyl)-ethyl amine

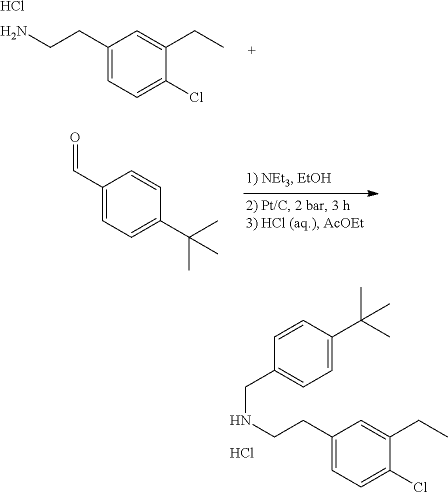

To a solution of 2-(4-Chloro-3-ethyl-phenyl)-ethylamine hydrochloride (10.0 g, 45.4 mmol, note 2) in AcOEt (90 ml) was added in a separation funnel water (34 ml) and NaOH (9.38 ml, 101.3 mmol, 2.23 eq.) and the phases were separated. The organic phase was washed with NaCl (aq.) solution (10 ml) whereas the aq. phases were washed with AcOEt (80 ml). The combined organic phases were dried over Na₂SO₄ and concentrated to a volume of ca. 80 ml. To this solution was added at RT 4-tert-butyl benzaldehyde (8.15 ml, 47.7 mmol, 1.05 eq.), the solution was stirred at RT overnight and the solvent was completely removed. The resulting yellow oil was dissolved in AcOEt (70 ml), the yellow solution of the imine was transferred into the autoclave and Pt/C (0.5 g, 0.128 mmol, 0.0028 eq., 5% w/w) was added under Ar. The autoclave was pressurized with 2 bar of H₂ and the reaction mixture was heated to 40° C. The reaction was run at 2 bar of H₂ pressure at 40° C. until full conversion was achieved. Afterwards, the pressure was carefully released, the dark suspension was filtered over dicalite and washed with AcOEt. Afterwards, the mother liquor was concentrated under vacuum to a volume of ca 20 ml and HCl (37%, 4.0 ml, 47.25 mmol, 1.04 eq.) was added whereupon white crystals were precipitating. The crystals were filtered off, washed with AcOEt and dried under vacuum at 50° C. until weight constancy to yield the title compound (15.4 g, 91.7% of theory) as white crystals (m.p.: 221.9-222.5° C.). ¹H-NMR (400 MHz, CDCl₃): δ 10.12 (br s, 1H), 7.40 (d, 2H), 7.38 (d, 2 H), 7.21 (d, 1H), 7.04 (d, 1H), 6.94 (dd, 1H), 3.92 (s, 2H), 3.18 (m, 2H), 2.99 (m, 2H), 2. 68 (q, 2H), 1.19 (s, 9H), 1.17 (t, 3H). MS (EI, Turbo spray): m/z=300 ([M+H]⁺, 100%). EA for C₂₁H₂₉Cl₂N: calcd. C, 68.85; H, 7.98; N, 3.82, Cl 19.35 found C, 68.74; H, 7.71; N, 3.84, Cl 19.47.

### Example 4.2

### Synthesis of (4-tert-butyl-benzyl)-[2-(4-chloro-3-ethyl-phenyl)-ethyl]-amine hydrochloride

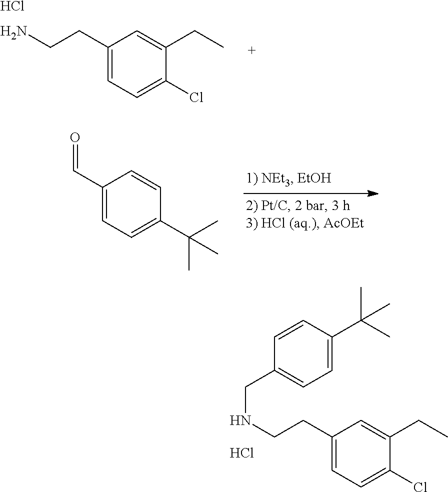

To a solution of 2-(4-Chloro-3-ethyl-phenyl)-ethylamine hydrochloride (70.0 g, 254.4 mmol) in EtOH (270 ml) was added at RT under stirring and under Ar NEt₃ (43.0 ml, 307.4 mmol, 1.21 eq.), the reaction mixture was stirred for 10 min and the addition funnel was rinsed with EtOH (40 ml). To this mixture was added over a period of 15 min 4-tert-butyl benzaldehyde (42.67 g, 255.1 mmol, 1.0 eq.), the addition funnel was rinsed with EtOH (40 ml) and the resulting reaction mixture was stirred overnight at RT. The yellow suspension was filtered over a fiber filter, washed with EtOH (350 ml) and the alcoholic solution of the imine was transferred into the autoclave. To this solution was added a slurry of Pt/C (4.7 g, 1.035 mmol, 0.0038 eq.) in EtOH (30 ml) and the autoclave was pressurized with 2 bar of H₂. The reaction was run at 2 bar of H₂ pressure at 40° C. for 1.5 h. Afterwards, the pressure was carefully released from the autoclave, the suspension was filtered through paper filter with a filtrox plate and speedex in order to remove the catalyst, the autoclave and the filter cake were washed with AcOEt (2000 ml) and the mixture was extracted with NaCl (aq., 2000 ml). The phases were separated, the organic phase was washed 3 times with in total NaCl (aq., 6000 ml) and the volume of the combined organic phases was reduced to ca. 850 ml. The mixture was filtered and AcOEt (850 ml) was added, the volume was adjusted to 850 ml as described above and the pH of the solution was adjusted to ca. 1 by addition of HCl (32.12 ml, 383.9, 1.51 eq.) whereupon the product started to precipitate. The white precipitate was filtered off, washed portionwise with in total AcOEt (in total 200 ml) and dried under vacuum at 50° C. overnight to yield the title compound (80.5 g, 86.0% of theory) as white crystals.

### Example 4.3

### Synthesis of (4-tert-butyl-benzyl)-[2-(4-chloro-3-ethyl-phenyl)-ethyl]-amine hydrochloride

A: Imine preparation: To a white suspension of 2-(4-Chloro-3-ethyl-phenyl)-ethylamine hydrochloride (100 g, 454 mmol, 1.0 eq.) in toluene (300 ml) was added NEt₃ (70.5 ml, 500 mmol, 1.1 eq.) during 15 min at RT. To this thick suspension was added at RT 4-tert-butyl benzaldehyde (83.1 ml, 490 mmol, 1.08 eq.), the reaction mixture was stirred for 1 h and NaCl (60 g) in water (543 g) was added. The mixture was stirred for 10 min and the phases were separated. The reactor was rinsed with toluene (70 ml) and the combined organic phases were used directly in the subsequent hydrogenation step.

B: Hydrogenation of imine: To Pt/C (5%, 11.1 g, 2.85 mmol, 0.0063 eq.) was added the above prepared imine solution in toluene at RT, followed by the addition of toluene (90 ml). The suspension was heated to 40° C., the autoclave was pressurized with 3.4 bar of H₂ and the mixture was hydrogenated for 2 h under vigorous stirring. The pressure was carefully released from the autoclave, the catalyst was filtered off, the autoclave and the filter were washed with toluene (140 ml) and to the corresponding filtrate was added toluene (1000 ml) and HCl (37%, 57 ml, 682 mmol, 1.5 eq.) to form a white suspension. The water was removed from the mixture upon heating the mixture on a Dean Stark apparatus at reflux temperature. The corresponding solution was cooled to 90° C. during 10 min and seeing crystals (100 mg) were added. The suspension was cooled to RT during 45 min and stirred for 1 h at this temperature. The crystals were filtered off, the reactor and the filter cake were washed with toluene (250 ml) and the dried under vacuum at 50° C. for 14 h to yield the title compound as white crystals (154 g, 91% of theory).

### Example 4.4

### Synthesis of (4-tert-butyl-benzyl)-[2-(4-chloro-3-ethyl-phenyl)-ethyl]-amine hydrochloride

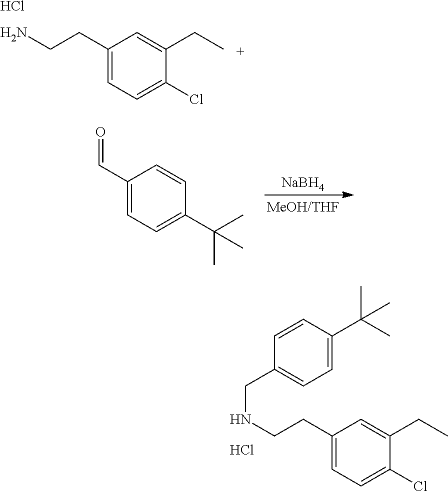

To a solution of 2-(4-Chloro-3-ethyl-phenyl)-ethylamine hydrochloride (36.3 g, 164.9 mmol) in MeOH (160 ml) was added at RT NaOMe (5.4 M, 31.7 g, 176.5 mmol, 1.07 eq.), the addition funnel was washed with MeOH (45 ml), the corresponding mixture was stirred for 10 min at RT, followed by the addition of 4-tert-butyl benzaldehyde (30.3 g, 181.2 mmol, 1.10 eq.). The addition funnel was rinsed with MeOH (20 ml) and the corresponding light yellow solution was stirred for 2 h at RT. Afterwards NaBH₄ (7.35 g, 186.5 mmol, 1.13 eq.) was added in three equal portions keeping the internal temperature between 22 and 30° C., the equipment was rinsed with MeOH (60 ml) and the white suspension was stirred at 30 to 22° C. for 1.5 h. The white suspension was quenched upon the addition of HCl (1 N, 140 ml, 140 mmol, 0.849 eq.) and stirred at RT for 1 h. The pH of the reaction mixture was adjusted to pH 8-9 upon the addition of NaHCO₃ (aq., 9%, 150 ml, 160.7 mmol, 0.975 eq.) and AcOEt (350 ml) was added. The bi-phasic mixture was filtered, the phases were separated and the organic phase was washed with water (250 ml) and NaCl solution (5%, 250 ml), whereas the aq. phase was washed with AcOEt (150 ml). The combined organic phases were treated with HCl (37%, 25.0 ml, 299.5 mmol, 1.82 eq.), forming a white suspension which was stirred overnight. The white suspension was stirred at RT for 2 h and in an ice bath for 1 h, the precipitate was filtered off, the crystals were washed portionwise with AcOEt (30 ml in total) and dried under vacuum until weight constancy to yield the title compound (50.7 g, 83.6% of theory) as fine white crystals.

### Example 4.5

### Synthesis of (4-iso-butyl-benzyl)-[2-(4-chloro-3-ethyl-phenyl)-ethyl]-amine hydrochloride

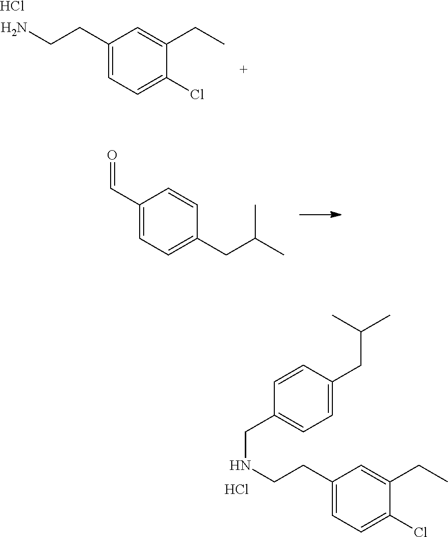

To a solution of 2-(4-Chloro-3-ethyl-phenyl)-ethylamine hydrochloride (4.0 g, 18.17 mmol) in toluene (40 ml) were added water (15 ml) and NaOH (3.75 ml, 40.52 mmol, 2.23 eq.). The organic phase was separated and washed with NaCl solution (sat., 30 ml), whereas the water phase was washed with toluene (50 ml). The combined organic phases were dried over Na₂SO₄ and concentrated under vacuum to dryness. The oily residue was dissolved in toluene (50 ml) and 4-iso-butyl benzaldehyde (3.26 ml, 19.1 mmol, 1.05 eq.) was added under Ar, the resulting slightly yellow solution was stirred for 2.5 h at RT and toluene was removed under vacuum. For complete water removal, toluene (200 ml in total) was added in two portions and removed under vacuum. The residual yellow oil was dissolved in MeOH (32 ml) and the resulting solution was added dropwise to an ice-cold suspension of NaBH₄ (687 mg g, 18.18 mmol, 1.0 eq.) in THF (16 ml). The corresponding light yellow reaction mixture was allowed to warm to RT, stirred for 17.5 h, quenched by addition of water (11 ml) at RT, and concentrated under vacuum. To the resulting water/oil suspension was added toluene (48 ml) and the mixture was stirred for 10 min. The organic phase was separated, washed twice with water (70 ml in total), dried over Na₂SO₄ and concentrated under vacuum. To the light yellow residue was added AcOEt (50 ml), the corresponding solution was treated with of HCl (2.76 ml, 33.07 mmol, 1.82 eq.) whereupon white crystals started to precipitate. The suspension was stirred at RT overnight, the precipitate was filtered off and dried under vacuum at 50° C. until weight constancy to yield the title compound (4.33 g, 65.0% of theory) as fine white crystals (m.p.=221.4-222-4° C.). ¹H-NMR (400 MHz, CDCl₃): δ 10.10 (br s, 2H), 7.47 (d, 2H), 7.22 (d, 2H), 7.12 (d, 1H), 7.01 (d, 1H), 6.91 (dd, 1H), 3.99 (s, 2H), 3.11 (m, 2H), 2.96 (m, 2H), 2.68 (q, 2H), 2.29 (d, 2H), 1.74 (m, 1 H), 1.18 (t, 3H), 0.82 (d, 6H). MS (EI): m/z=330 ([M+H]⁺, 100%).

### Example 4.6

### Synthesis of (4-tert-Butyl-benzyl-[2-(3-fluoro-5-trifluoromethylphenyl)-ethyl]-amine hydrochloride

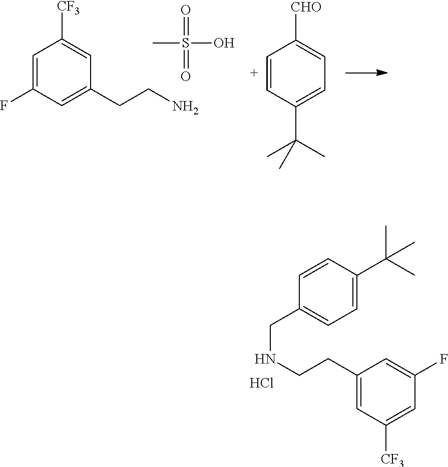

To a solution of 2-(3-Fluoro-5-trifluoromethylphenyl)-ethylamine mesylate (64.0 g, 211.0 mmol) in MeOH (640 ml) was added NaOMe (39.2 ml, 211.0 mmol, 1.00 eq.), the addition funnel was washed with MeOH (145 ml), the corresponding mixture was stirred for 10 min at RT, followed by sequential addition of 4-tert-butyl benzaldehyde (34.2 g, 211.0 mmol, 1.00 eq.), acetic acid (12.6 ml, 219.4 mmol, 1.04 eq.) and Na(OAc)₃BH (96.0 g, 430.4 mmol, 2.04 eq.) at 25-30° C. The corresponding suspension was stirred at RT for 1 h, followed by addition of HCl (37%, 61.7 ml, 738.5 mmol, 3.5 eq.) in water (1500 ml) whereupon crystallization was initiated. The resulting suspension was stirred for 30 min in the ice bath, the precipitate was filtered off, washed with water and dried under vacuum at 60° C. to yield the title compound (83.0 g, 84.2% of theory) as fine white crystals. ¹H-NMR (400 MHz, CDCl₃): δ 9.52 (br s, 2H), 7.60-7.41 (m, 7H), 4.12 (br s, 2H), 3.49-3.09 (m, 4H), 1.28 (s, 9H). MS (EI, turbo spray): m/z=354 ([M+H]⁺, 100%).

### Example 4.7

### Synthesis of (4-tert-Butyl-benzyl-[2-(3-fluoro-5-trifluoromethylphenyl)-ethyl]-amine mesylate

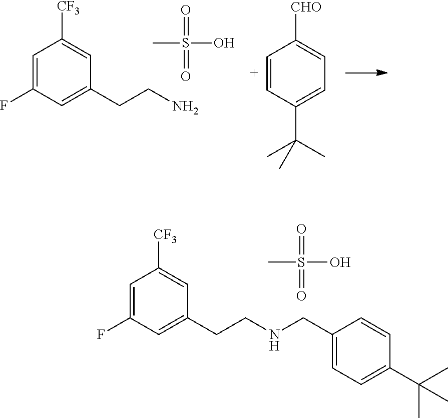

To a solution of 2-(3-Fluoro-5-trifluoromethylphenyl)-ethylamine mesylate (30.0 g, 98.92 mmol) in toluene (270 ml) was added water (102 ml) and NaOH (20.4 ml, 220.6 mmol, 2.23 eq.). The organic phase was separated and washed with NaCl solution (100 ml), whereas the water phase was washed with toluene (200 ml). The combined organic phases were dried over Na₂SO₄ and concentrated under vacuum to a total volume of ca. 300 ml. To this solution was added under Ar 4-tert-butyl benzaldehyde (17.7 ml, 103.9 mmol, 1.05 eq.), the resulting slightly yellow solution was stirred for 2 h at RT and toluene was removed under vacuum. The residual yellow oil was dissolved in MeOH (240 ml) and the resulting solution was added dropwise to an ice-cold suspension of NaBH₄ (3.74 g, 98.92 mmol, 1.0 eq.) in THF (120 ml). The corresponding light yellow reaction mixture was allowed to warm to RT, stirred for 1 h, quenched by addition of water (60 ml) at RT and concentrated under vacuum. To the resulting water/oil suspension was added toluene (375 ml) and the mixture was stirred for 10 min. The organic phase was separated, washed twice with water and was concentrated under vacuum. To the light yellow residue was added MeOH (150 ml), the corresponding solution was cooled to 0-5° C. in an ice bath, then a solution of methane sulfonic acid (7.8 ml, 118.7 mmol, 1.2 eq.) dissolved in water (120 ml) was added dropwise within 15 min. During the addition white crystals started to precipitate. To dissolve these crystals, DCM (300 ml) was added and the phases were separated. The aq. phase was washed with DCM (100 ml), whereas the organic phase was washed with water (100 ml). The combined organic phases were dried over Na₂SO₄ and the solvents were exchanged on the rotary evaporator with tert-butyl methyl ether (300 ml). In the course of this operation a precipitate started to form. The suspension was cooled in an ice bath for 30 min, the precipitate was filtered off and dried under vacuum to yield the title compound (38.5 g, 86.2% of theory) as fine white crystals (m.p.: 228.8-230.8° C.). ¹H-NMR (400 MHz, CDCl₃): δ 9.23 (br s, 2H), 7.45-7.38 (m 4H), 7.23 (s, 1H), 7.20-7.12 (m, 2H), 4.12 (s, 2H), 3.2 (m, 2H), 3.04 (m, 2H), 2.79 (s, 3H), 1.25 (s, 9H). MS (EI, turbo spray): m/z=354 ([M+H]⁺, 100%). EA for C₂₁H₂₇F₄NO₃S: calcd. C, 56.11; H, 6.05; N, 3.12. Found C, 55.98; H, 6.05; N, 3.11.

### Example 4.8

### (4-iso-Butyl-benzyl-[2-(3-fluoro-5-trifluoromethylphenyl)-ethyl]-amine hydrochloride

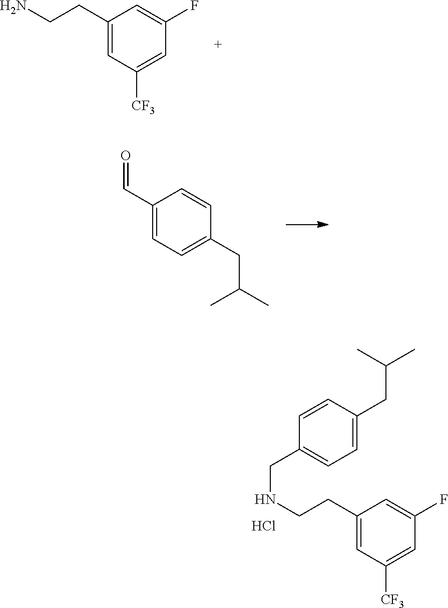

To a solution of 2-(3-Fluoro-5-trifluoromethylphenyl)-ethylamine (0.77 g, 3.46 mmol) and 4-iso-butyl benzaldehyde in MeOH (10 ml) was added potassium carbonate (0.48 g, 3.46 mmol, 1.0 eq.), the reaction mixture was stirred for 30 min at RT and was then heated to 50° C. for 2.5 h. The reaction mixture was cooled to RT, NaBH₄ (0.196 g, 5.19 mmol, 1.5 eq.) was added and the reaction mixture was heated to 50° C. for 2.5 h. The white suspension was cooled to RT and HCl (2M, 3.46 ml, 6.91 mmol, 2 eq.) was added carefully. The solvents were removed under vacuum and the residue was taken up in AcOEt/water (1:1, 40 ml). The phases were separated, the aq. phase was washed with AcOEt (20 ml), whereas the organic phase was washed with brine. The combined organic phases were dried over Na₂SO₄ and concentrated under vacuum to give a yellow oil. This oil was purified by flash chromatography with AcOEt/n-heptane (1:1) as eluent yielding the title compound (1.00 g, 77.8% of theory) as colorless oil. ¹H-NMR (300 MHz, CDCl₃): δ 7.27-7.05 (m, 7H), 3.77 (s 2H), 2.88 (m, 4H), 2.45 (m, 2H), 1.85 (m, 1H), 0.89 (d, 6H).

### Example 4.9

### [1-(4-tert-Butyl-phenyl)-meth-(E)-ylidene]-[2-(4-chloro-3-ethyl-phenyl-ethyl]-amine

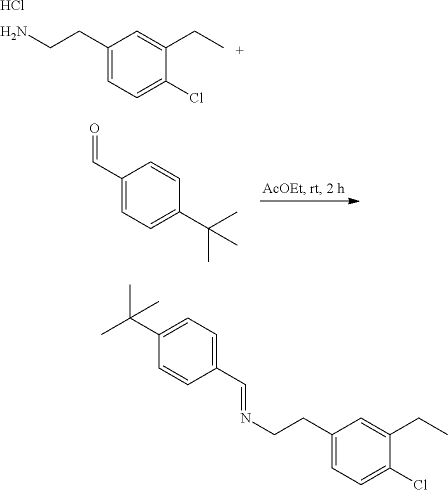

To a solution of 2-(4-Chloro-3-ethyl-phenyl)-ethylamine hydrochloride (2.0 g, 9.09 mmol) in AcOEt (20 ml) was added in a separation funnel water (20 ml) and NaOH (2.0 ml, 21.6 mmol, 2.38 eq.) and the phases were separated. The aq. phases were washed with AcOEt (20 ml) and the combined organic phases were dried over Na₂SO₄ and evaporated under vacuum. The oily residue was dissolved in AcOEt (20 ml) and 4-tert-butyl benzaldehyde (1.63 ml, 9.75 mmol. 1.07 eq.) was added. The reaction mixture was stirred at ambient temperature for 2 h and the solvent was removed under vacuum to yield the title compound as a light yellow oil in quantitative yield. ¹H-NMR (400 MHz, CDCl₃): δ 8.11 (s, 1H), 7.62 (m, 2H), 7.42 (m, 2H), 7.22 (d, 1H), 7.08 (d, 1H), 6.95 (m, 1H), 3.81 (m, 2H), 2.95 (m, 2H), 2.68 (q, 2H), 1.33 (s, 9 H), 1.16 (t, 3H).

### Example 5.1

### Synthesis of N-(4-tert-butyl-benzyl)-3-chloro-N-[2-(4-chloro-3-ethyl-phenyl)-ethyl]-2-fluoro-5-trifluoromethyl-benzamide

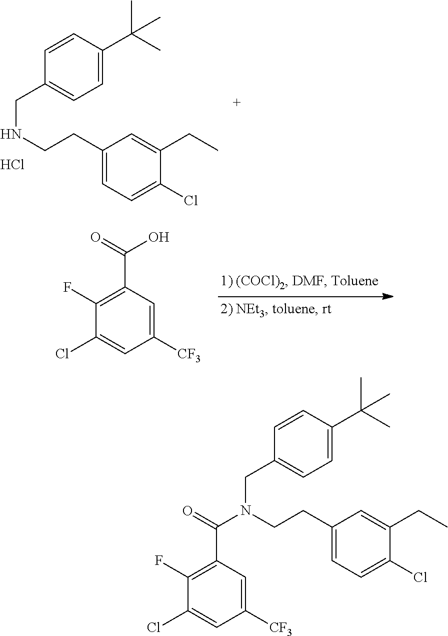

To a solution of 2-Fluoro-3-chloro-5-trifluoromethyl-benzoic acid (33.9 g, 135.6 mmol, 1.2 eq.) in toluene (450 ml) was added under Ar in apparatus A at RT and under vigorous stirring DMF (1.7 ml, 22.0 mmol, 0.195 eq.) and oxalyl chloride (16.0 g, 123.5 mmol, 1.09 eq.) and the reaction mixture was stirred for 30 min. This reaction mixture was added at an internal temperature of 20-35° C. to a previously prepared suspension (in Apparatus B) of (4-tert-butyl-benzyl)-[2-(4-chloro-3-ethyl-phenyl)-ethyl]-amine hydrochloride (41.5 g, 113.0 mmol), toluene (600 ml) and triethyl amine (66.7 ml, 467.8 mmol, 4.22 eq.). The Apparatus A was rinsed with toluene (20 ml), the white suspension was stirred for 1 h at RT and the filter cake was washed portionwise with toluene (300 ml). The filtrate was extracted sequentially with water (500 ml), HCl (1 M, 500 ml), NaHCO₃ (saturated, 500 ml) and water (500 ml) and the organic phase was evaporated to dryness under vacuum. The oil was dissolved at 45° C. in 2-propanol (200 ml) and water (13.5 ml) was added. The solution was cooled to 25° C. within 2 h, seeding crystals were added and at RT water (27 ml) was added. The suspension was stirred for 5 h at RT and 1 h in an ice bath, the white crystals were filtered off, washed portionwise with ice-cold 2-propanol/water (4:1) and the crystals were dried under vacuum at 50° C. to give the title compound (57.3 g, 91.2% of theory) as white crystals. MS (EI): m/z=554 ([M+H]⁺, 100%).

### Example 5.2

### Synthesis of N-(4-tert-butyl-benzyl)-3-chloro-N-[2-(4-chloro-3-ethyl-phenyl)-ethyl]-2-fluoro-5-trifluoromethyl-benzamide

Sequence A: To a suspension of 2-Fluoro-3-chloro-5-trifluoromethyl-benzoic acid (62.4 g, 215 mmol) in Toluene (364 ml) was added under Ar in a reactor 1 at 20-30° C. and under vigorous stirring N-Formylpiperidine (2.8 ml, 25.1 mmol, 0.1 eq.). The mixture was stirred for 10 min at 20-30° C. whereupon a colorless solution was obtained. To this solution was added at 20-30° C. oxalyl chloride (33.5 g, 264 mmol, 1.05 eq.) over a period of 30 min, the addition funnel was rinsed with toluene (20 ml) and the reaction mixture was stirred at 20-30° C. for 1 h. Then ca. 80 ml of toluene were removed under reduced pressure (60-100 mbar) at 30-45° C. The reactor was rinsed with toluene (40 ml) and this solution was used for the subsequent amine coupling (sequence B) without further purification. Sequence B: To a suspension of (4-tert-butyl-benzyl)-[2-(4-chloro-3-ethyl-phenyl)-ethyl]-amine hydrochloride (80.0 g, 218.0 mmol) in toluene (260 ml) was added triethylamine (44.2 g, 437 mmol, 2 eq.) dropwise within 5-10 min at an inside temperature of 20-25° C. and the white suspension was stirred for 15 min at 20-25° C. To this thick suspension was added dropwise at 20-35° C. 2-Fluoro-3-chloro-5-trifluoromethyl-benzoic acid in toluene (preparation described under sequence A, 315 g, 215 mmol, 1.15 eq.) over a period of 45 min. The addition funnel was rinsed with toluene (20 ml) and the reaction mixture was stirred for 1 h at 20-25° C. Then NaOH (3M, 400 ml) was added and the mixture was stirred for 10 min. The phases were separated and the organic phase was treated with water (400 ml) and stirred for 10 min. The phases were separated and the volume of the organic phase was adjusted to 140 ml under reduced pressure (60 mbar, 35-45° C.). To the toluene solution of the crude product was added ethanol (320 ml) at 40° C. and 300 ml of the toluene/ethanol mixture were removed under reduced pressure (60 mbar, 30-45° C.). Afterwards, ethanol was added (460 ml) and the mixture was polish filtered and the filter was rinsed with ethanol (83 ml). The solution was then cooled to 14-16° C. and seeding crystals (3.6 g, 3 w/w %) suspended in a ethanol/water mixture (36 ml, ratio 80:20) were added. The white suspension was cooled to 4-6° C. within 2 h and stirred at this temperature for 3 h. Then ethanol/water (290 g, ratio 50:50) was added during 45 min, the white precipitate was filtered off and washed with ethanol/water (100 g, ratio 80:20). The obtained crystals were dried (10 mbar, 50° C.) to give the title compound (109.3 g, 84.1% Yield of theory).

### Example 5.3

### Synthesis of N-(4-tert-butyl-benzyl)-3-chloro-N-[2-(4-chloro-3-ethyl-phenyl)-ethyl]-2-fluoro-5-trifluoromethyl-benzamide

The product to be re-crystallized was synthesized as described in Example 5.2. 20 g of the product was dissolved in the corresponding alcohol at 40° C. to give a 20 w/w % solution, then the solution was cooled to 15° C., seeding crystals (3-5 w/w %) were added and the temperature was kept at 15° C. for 5-7 h. The reaction mixture was cooled to 5° C. within 2 h and stirred at 5° C. for 3 h. Then 20-30 v % of water was added over a period of 45 min. The obtained crystals were filtered off with suction, washed with a mixture of alcohol/water (ratio 70:30 or 80:20) and dried under vacuum until weight constancy. According to this procedure the experiments in Table 5.3 were performed.

TABLE 5.3

| | Solvent | Seeding crystals | Water content | |
|---|---|---|---|---|
| Exp. Nr. | (alcohol) | (Amount in % w/w) | (%) | Yield (%) |
| 1 | methanol | 3 | 20 | 98.2 |
| 2 | ethanol | 3 | 20 | 93.4 |
| 3 | 2-propanol | 3 | 20 | 93.1 |
| 4 | n-propanol | 5 | 30 | 97.5 |

### Example 5.4

### Synthesis of N-(4-tert-butyl-benzyl)-3-chloro-N-[2-(4-chloro-3-ethyl-phenyl)-ethyl]-2-fluoro-5-trifluoromethyl-benzamide

The product to be re-crystallized was synthesized as described in Example 5.2. 30 g of the product was dissolved in the acetonitrile/water 90:10 at 40° C. to give a 30% (w/w) solution, then the solution was cooled to 15° C., seeding crystals (5 w/w %) were added and the temperature was kept at 15° C. for 5-7 h. The reaction mixture was cooled to 5° C. within 2 h, stirred at 5° C. for 3 h and water was added over a period of 45 min to achieve a acetonitrile/water ratio of 70:30. The obtained crystals were filtered off with suction, washed with a mixture of acetonitrile/water (ratio 70:30) and dried under vacuum until weight constancy to give the title compound (28.9 g, 95.6% yield of theory).

### Example 5.5

### Synthesis of N-(4-tert-butyl-benzyl)-3-chloro-N-[2-(4-chloro-3-ethyl-phenyl)-ethyl]-2-fluoro-5-trifluoromethyl-benzamide

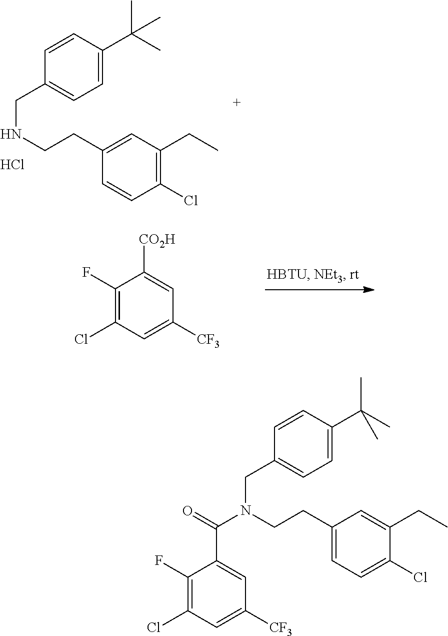

To a suspension of (4-tert-butyl-benzyl)-[2-(4-chloro-3-ethyl-phenyl)-ethyl]-amine hydrochloride (2.0 g, 5.46 mmol) and 3-chloro-2-fluoro-5-trifluoromethyl benzoic acid (1.37 g, 5.46 mmol, 1.0 eq.) and HBTU (2.54 g, 6.55 mmol, 1.2 eq.) in THF (30 ml) was added under Ar and with stirring NEt₃ (1.68 ml, 12.06 mmol, 2.21 eq.) and the suspension was stirred for 17 h at RT. THF was exchanged with hexane (30 ml) under vacuum, MeOH (32 ml) and water (8 ml) was added and the mixture was stirred for 5 min at RT. The organic phase was separated, washed four times with MeOH/water 4:1, whereas the aqueous phase was washed with hexane (40 ml). The combined organic phases were evaporated to dryness and the corresponding clear light yellow oil was taken up in 2-propanol/water (85:15 w/w, 40 ml), dissolved at 40° C. and cooled to RT. At 10° C. seeding crystals (100 mg), the suspension was stirred at 4-6° C. overnight and 4 h at −10° C. Then the formed crystals were filtered off and dried under vacuum at RT until weight constancy to yield the title compound (2.26 g, 73.8% of theory) as white crystals.

### Example 5.6

### Synthesis of N-(4-iso-butyl-benzyl)-3-chloro-N-[2-(4-chloro-3-ethyl-phenyl)-ethyl]-2-fluoro-5-trifluoromethyl-benzamide

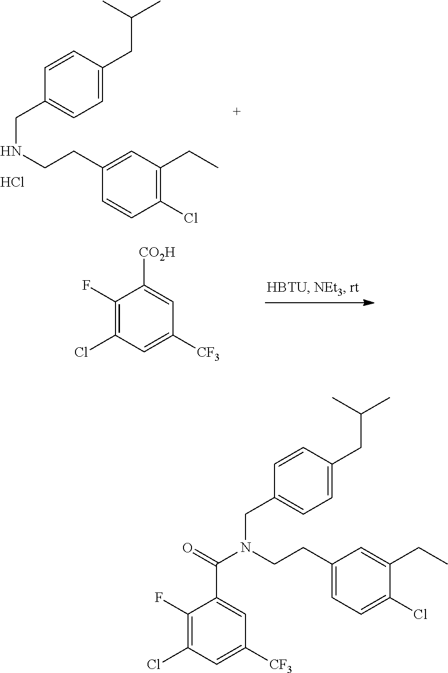

To a suspension of (4-iso-butyl-benzyl)-[2-(4-chloro-3-ethyl-phenyl)-ethyl]-amine hydrochloride (4.0 g, 10.92 mmol) and 3-chloro-2-fluoro-5-trifluoromethyl benzoic acid (3.03 g, 12.12 mmol, 1.11 eq.) and HBTU (5.63 g, 14.54 mmol, 1.33 eq.) in THF (60 ml) was added under Ar and with stirring NEt₃ (3.72 ml, 26.73 mmol, 2.45 eq.), the suspension was stirred for 18.5 h at RT and THF was removed under vacuum. The brownish residue was taken up under stirring in hexane (60 ml), MeOH (64 ml) and water (16 ml) was added and the mixture was stirred for 5 min at RT. The organic phase was separated, washed four times with MeOH/water 4:1 (100 ml in total), whereas the aqueous phase was washed with hexane (40 ml). The combined organic phases were dried over Na₂SO₄, the solvents were evaporated and the corresponding residue was dried under vacuum until weight constancy to yield the title compound (6.17 g, 98.9% of theory) as orange oil. MS (EI): m/z=554 ([M+H]⁺, 100%).

### Example 5.7

### Synthesis of N-(4-tert-butyl-benzyl)-3-N-[2-(4-chloro-3-ethyl-phenyl)-ethyl]-2-fluoro-5-trifluoromethyl-benzamide

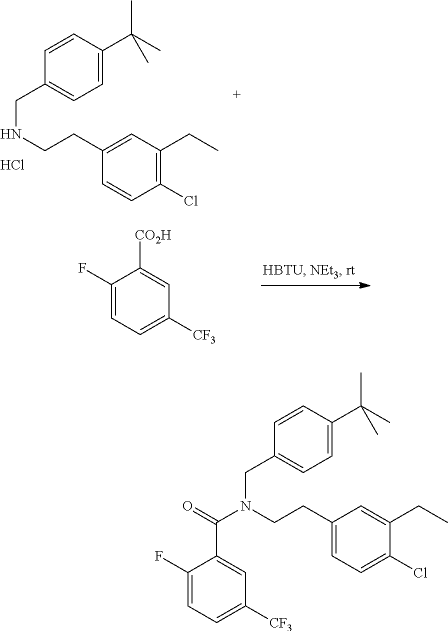

To a suspension of (4-tert-butyl-benzyl)-[2-(4-chloro-3-ethyl-phenyl)-ethyl]-amine hydrochloride (1.73 g, 4.71 mmol) and 2-fluoro-5-trifluoromethyl benzoic acid (1.0 g, 4.71 mmol, 1.0 eq.) and HBTU (2.19 g, 5.65 mmol, 1.2 eq.) in THF (26 ml) was added under Ar and with stirring NEt₃ (1.45 ml, 10.41 mmol, 2.21 eq.), the suspension was stirred for 22 h at RT and THF was removed under vacuum. The brownish residue was taken up under stirring in hexane (26 ml), MeOH (28 ml) and water (7 ml) was added and the mixture was stirred for 5 min at RT. The organic phase was separated, washed four times with MeOH/water 4:1 (100 ml in total), whereas the aq. phase was washed with hexane (40 ml). The combined organic phases were dried over Na₂SO₄ and the solvents were evaporated and the corresponding residue was dried under vacuum until weight constancy to yield the title compound (2.39 g, 93.1% of theory) as orange oil. MS (EI): m/z=520 ([M+H]⁺, 100%).

### Example 5.8

### Preparation of 3-chloro-2-fluoro-5-trifluoromethyl benzoic acid chloride with N,N-diphenyl formamide as catalyst

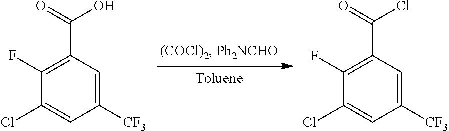

A suspension of 3-chloro-2-fluoro-5-trifluoromethyl benzoic acid (30.6 g, 126 mmol) and N,N-diphenyl formamide (2.51 g, 12.6 mmol) in toluene (182 mL) was treated at 20 to 30° C. within 30 minutes with oxalyl chloride (17.0 g, 133 mmol) and the resulting mixture was stirred at 25° C. for 4 hours. Upon complete conversion approx. 80 mL of toluene were distilled off to remove the excess of oxalyl chloride. The clear residue was directly used in the amine coupling step as described in Example 5.2 (sequence B) without further purification.

### Example 5.9

### Synthesis of N-(4-tert-Butyl-benzyl)-3-chloro-2-fluoro-N-[2-(3-fluoro-5-trifluoromethyl-phenyl)-ethyl]-5-trifluoromethyl-benzamide

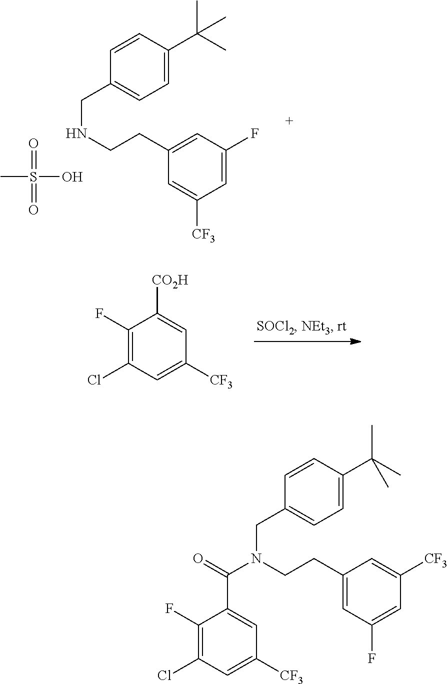

### Section A: Preparation of Acid Chloride

To a solution of 3-chloro-2-fluoro-5-trifluoromethyl benzic acid (5.5 g, 22.81 mmol) in toluene (75 ml) was added dropwise thionyl chloride (1.8 ml, 25.03 mmol, 1.1 eq.) and the resulting solution was heated at reflux temperature for 3 h. After cooling to RT, toluene and the excess of thionyl chloride were removed under vacuum to give a colorless viscous oil, which was used without any further purification (section B) and dissolved in THF (25 ml).

### Section B: Free the Amine and Coupling of the Two Fragments

To a solution of (4-tert-Butyl-benzyl-[2-(3-fluoro-5-trifluoromethylphenyl)-ethyl]-amine mesylate (10.0 g, 22.14 mmol, 0.97 eq.) in DCM (100 ml) was added a solution of NaOH (32%, 10 ml, 108 mmol, 4.88 eq.) in water (100 ml). The organic phase was separated and washed with water (100 ml) whereas the aq. phases were washed with DCM (100 ml). The combined organic phases were dried over Na₂SO₄, the solids were filtered off and the filtrate was treated under vacuum with THF (100 ml) to replace the solvent.

To the THF solution of 4-tert-Butyl-benzyl-[2-(3-fluoro-5-trifluoromethylphenyl)-ethyl]-amine was added slowly NEt₃ (6.7 ml, 48.03 mmol, 2.17 eq.) and the resulting solution was stirred for 20 min at RT, then the 3-Chloro-2-fluoro-5-trifluoromethyl-benzoyl chloride solution was slowly added via addition funnel. The resulting suspension was stirred at RT for 1.5 h, the precipitate was filtered off, washed with THF (20 ml) and the solvent was removed under vacuum to give a colorless viscous oil. This residual oil was dissolved under vigorous stirring in a mixture of n-hexane (150 ml), MeOH (200 ml) and water (50 ml). The organic phase was separated, and washed four times with a total amount of MeOH/water (4:1, 625 ml), whereas the aq. phases were washed with n-hexane (150 ml). The combined organic phases were dried over Na₂SO₄, the solids were filtered off and the filtrate was dried under vacuum to yield the crude product (12.12 g, 94.8% of theory) as a slightly yellow oil. The crude product was dissolved at 40° C. in 2-propanol/water (85:15, 80 ml), and the solution was slowly cooled to 10° C. (within 1 h). At this temperature seeding crystals (60 mg) were added, and the mixture was allowed to stir over the weekend (70 h in total) in the fridge (6-8° C.). Afterwards, the suspension was cooled to −10° C. and stirred at this temperature for 4 h. The precipitate was filtered off, washed with small amount of 2-propanol/water (85:15) and dried under vacuum at RT for 4 h until weight constancy to yield the title compound (9.8 g, 76.3% of theory) as white crystals (m.p.: 55.1-55.6° C.). MS (EI): m/z=578 ([M+H]⁺, 100%). EA for C₂₈H₂₄ClF₈NO: calcd. C, 58.19; H, 4.19; N, 2.42; O, 2.77. Found C, 58.41; H, 4.09; N, 2.33; O, 2.74.

### Example 5.10

### Synthesis of N-(4-tert-Butyl-benzyl)-3-chloro-2-fluoro-N-[2-(3-fluoro-5-trifluoromethyl-phenyl)-ethyl]-5-trifluoromethyl-benzamide

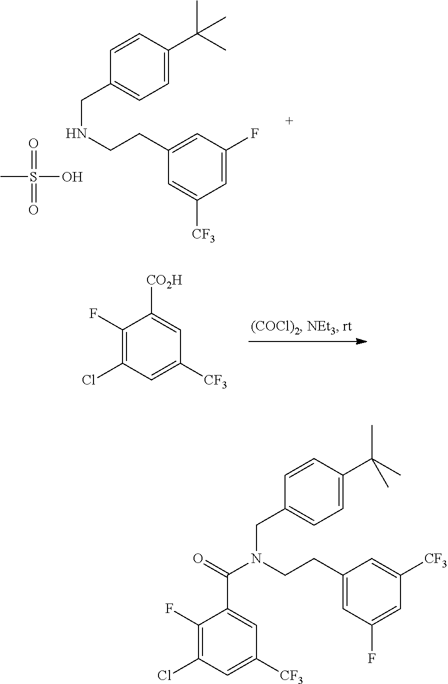

### Section A: Preparation of Acid Chloride

To a solution of 3-chloro-2-fluoro-5-trifluoromethyl benzic acid (16.2 g, 66.52 mmol, 1.1 eq. as to sec. amine in section B) in toluene (150 ml) was added at RT over a period of 5 min DMF (690 μl) and oxalyl chloride (8.3 g, 62.77 mmol, 1.05 eq. as to sec. amine in section B), the addition funnel was rinsed with toluene (6 ml) and the reaction was stirred for 30 min. This acid chloride solution was used without any further purification (see section B).

### Section B: Free the Amine and Coupling of the Two Fragments

To a solution of (4-tert-Butyl-benzyl-[2-(3-fluoro-5-trifluoromethylphenyl)-ethyl]-amine mesylate (26.97 g, 60.00 mmol, 1.00 eq.) in toluene (325 ml) and triethyl amine (35.2 ml, 252.9 mmol, 4.21 eq.) was added at 20-30° C. via cannula the above prepared acid chloride solution within 5 min, and the equipment was rinsed with toluene (9 ml) and the reaction mixture was stirred for 1 h at RT. The suspension was filtered, the filter cake was washed with toluene (100 ml), water (100 ml) was added and the bi-phasic mixture was stirred for 10 min. The phase was separated, the organic phase was sequentially washed with water (100 ml) HCl (25%, 25 ml, 191.8 mmol, 3.2 eq.) in water (150 ml), water (100 ml), NaHCO₃ (40.32 g, 480.0 mmol, 8.0 eq.) in water (100 ml) and water (100 ml). From the combined organic phases 550 ml of solvent was distilled off under vacuum, 2-propanol (200 ml) was added and the solvent was exchanged resulting in a solution of a weight of 50 g. To this solution was added 2-propanol (206 ml) and water (31.2 ml), the corresponding solution was cooled to 0° C., at 10° C. seeding crystals (20 mg) were added and the mixture was stirred at 0° C. for 5 h. Over a period of 10 min at 0-5° C. water (45.05 ml) was added and the obtained crystals were filtered off, washed with of 2-propanol/water (1:1, 60 ml) and dried under vacuum at RT for 48 h until weight constancy to yield the title compound (31.4 g, 90.4% of theory) as white crystals.

### Example 5.11

### Synthesis of N-(4-tert-Butyl-benzyl)-3-chloro-2-fluoro-N-[2-(3-fluoro-5-trifluoromethyl-phenyl)-ethyl]-5-trifluoromethyl-benzamide

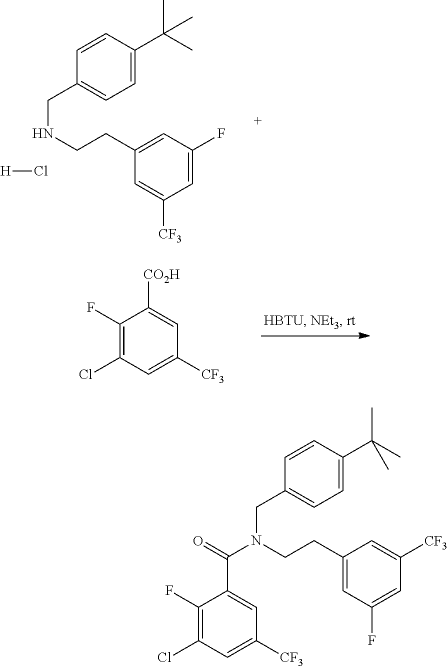

To a suspension of (4-tert-Butyl-benzyl-[2-(3-fluoro-5-trifluoromethylphenyl)-ethyl]-amine hydrochloride (70.0 g, 143.54 mmol), 3-chloro-2-fluoro-5-trifluoromethyl-benzoic acid (34.8, 143.54, 1.0 eq.), and HBTU (65.4 g, 172.3 mmol, 1.2 eq.) in THF (960 ml) was added under Ar and with stirring NEt₃ (44.2 ml). The suspension was stirred for 20 h at RT, then water (280 ml) was added. The solvent was partially evaporated and to the resulting residue was added at RT hexane (980 ml), MeOH (1100 ml) and water (70 ml). The phases were separated and the organic phase was extracted in four portions with a mixture MeOH (2800 ml in total), water (700 ml in total) and HCl (18.0 ml, 215.4 mmol, 1.5 eq.). The aq. phases were washed with a total amount of hexane (850 ml), whereas the combined organic phases were washed with a solution of NaHCO₃ (20.5 g, 9244.1 mmol, 1.7 eq.) in water (400 ml) and with water (200 ml). The organic phase was filtered for particle removal and concentrated under vacuum to a volume of ca. 200 ml. The solvent was exchanged under vacuum with 2-propanol (400 ml). The resulting solution was diluted with 2-propanol (291 ml) and water (59.3 ml) and cooled to −10° C. At this temperature seeding crystals (650 mg) were added and the suspension was stirred at 5° C. for 5 h. Afterwards, over a period of 10 min water (85 ml) was added and the white suspension was stirred over the week end. The formed crystals were filtered off and dried under vacuum at 25° C. until weight constancy to yield the title compound (56.6 g, 68.1% of theory) as white crystals.

### Example 5.12

### Synthesis of N-(4-tert-Butyl-benzyl)-3-chloro-2-fluoro-N-[2-(3-fluoro-5-trifluoromethyl-phenyl)-ethyl]-5-trifluoromethyl-benzamide

To a suspension of (4-tert-Butyl-benzyl-[2-(3-fluoro-5-trifluoromethylphenyl)-ethyl]-amine hydrochloride (750 mg, 1.92 mmol) and 3-chloro-2-fluoro-5-trifluoromethyl benzoic acid (476 mg, 1.96 mmol, 1.02 eq.) in AcOEt (10 ml) was added dropwise under Ar and with stirring NEt₃ (2.41 ml, 17.32 mmol, 9 eq.). The suspension was cooled to −10° C. followed by the addition of propanephosphonic acid anhydride (1.80 ml, 3.02 mmol, 1.57 eq.) and the reaction mixture was allowed to reach RT and was stirred overnight. The suspension was filtered and washed with AcOEt (10 ml) and water (20 ml) was added to the filtrate. The phases were separated and the organic phase was washed with water (10 ml) and brine (10 ml). The combined organic phases were dried over Na₂SO₄, filtered, the solvent was removed under vacuum and the residue was dried under vacuum at 50° C. until weight constancy to yield the title compound (1.14 g, 99.7% of theory) as light brown oil.

### Example 5.13

### Synthesis of N-(4-tert-Butyl-benzyl)-3-chloro-2-fluoro-N-[2-(3-fluoro-5-trifluoromethyl-phenyl)-ethyl]-5-trifluoromethyl-benzamide

To a suspension of (4-tert-Butyl-benzyl-[2-(3-fluoro-5-trifluoromethylphenyl)-ethyl]-amine hydrochloride (750 mg, 1.92 mmol) in AcOEt (10 ml) was added under Ar and with stirring NEt₃ (0.4 ml, 2.89 mmol, 1.5 eq.) and the suspension was stirred for 30 min at RT. Afterwards EDCI (414 mg, 2.12 mmol, 1.1 eq.) and HOBt (53.1 mg, 0.39 mmol, 0.2 eq.) were added, the reaction mixture was stirred for 1 h at RT followed by the addition of 3-chloro-2-fluoro-5-trifluoromethyl benzoic acid (476 mg, 1.96 mmol, 1.02 eq.). The reaction mixture was heated to 40° C. and stirred for 1 h at this temperature. The suspension was cooled to RT and water (10 ml) was added, forming a solution. The phases were separated and the organic phase was washed with water (10 ml) and brine (10 ml). The combined organic phases were dried over Na₂SO₄, filtered, the solvent was removed under vacuum and the residue was dried under vacuum at 50° C. until weight constancy to yield the title compound (1.18 g, 98.9% of theory) as yellow oil.

What is claimed:

1. A process for the preparation of a benzamide derivative of formula (I): 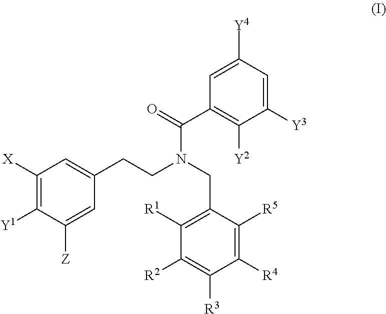
wherein
X and Z are each independently selected from the group consisting of hydrogen, halogen, (C₁-C₆)alkyl, halo(C₁-C₆)alkyl, C₃-C₇cycloalkyl and halo(C₁-C₆)alkoxy;
Y¹, Y² and Y³ are each independently hydrogen or halogen atoms;
Y⁴ is (C₁-C₆)alkyl or (C₁-C₃)perhaloalkyl;
R¹, R², R⁴ and R⁵ are each independently selected from the group consisting of hydrogen, halogen, (C₁-C₆)alkyl, and (C₁-C₆)alkoxy;
R³ is selected from the group consisting of halogen, (C₁-C₆)alkyl, hydroxy(C₁-C₆)alkyl, —Si((C₁-C₆)alkyl)₃ (C₁-C₆)alkoxy, halo(C₁-C₆)alkoxy, halo(C₁-C₆)alkyl, C₃-C₇cycloalkyl, phenyl, benzyl, and —NR¹¹R¹², wherein R¹¹ and R¹² independently from each other are selected from hydrogen, C₁-C₆alkyl, and phenyl, and —C(O)—OR¹³, wherein R¹³ is hydrogen or C₁-C₆alkyl;
which comprises reacting compound of formula (II) 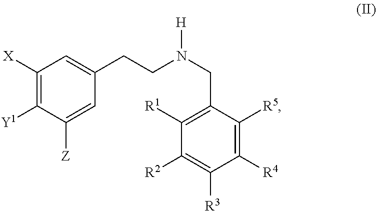
wherein X, Z, Y¹, R¹, R², R³, R⁴ and R⁵ are as defined above, with a compound of formula (III) 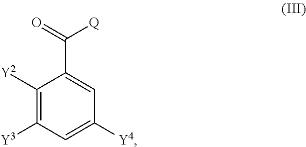
wherein Y², Y³ and Y⁴ are as defined above and Q is hydroxy or chloride, in the presence of a tertiary amine with the proviso that when Q is hydroxy, a coupling agent is present.

2. A process for the preparation of the compound of formula (II) 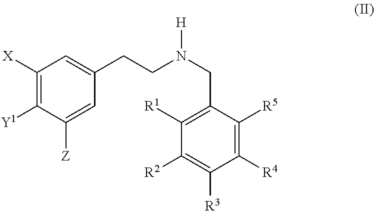
wherein X, Z, Y¹, R¹, R², R³, R⁴ and R⁵ are as defined in claim 1, which comprises
a) reacting a compound of formula (IV) 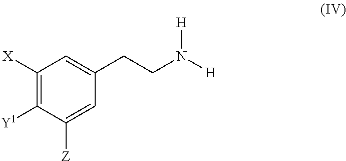
wherein X, Z and Y¹ are as defined in claim 1 with a compound of formula (V) 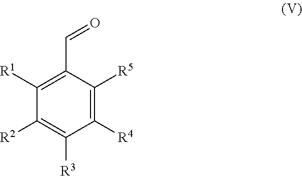
wherein R¹, R², R³, R⁴ and R⁵ are as defined in claim 1 to obtain an imine derivative of formula (VI), 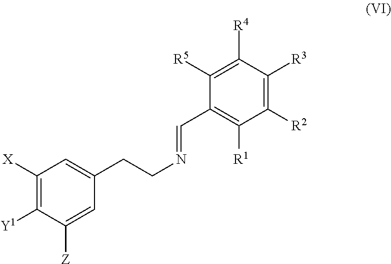
wherein X, Z, Y¹, R¹, R², R³, R⁴ and R⁵ are as defined in claim 1;
b) reacting the said imine derivative with H₂ in the presence of a metal catalyst or with a reducing agent to obtain a compound of formula (II) with the proviso that when LiAlH₄ or Pd/C is used, none of the groups X, Z, Y¹, R¹, R², R³, R⁴ and R⁵ are chloride.

3. A process for the preparation of compound of formula (VII) 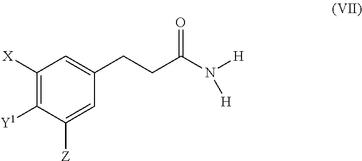
wherein X, Z and Y¹ are as defined in claim 1, which comprises reacting a compound of formula (VIII) 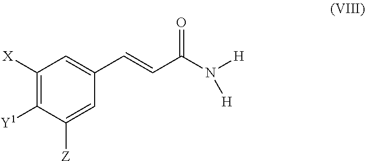
wherein X, Z and Y¹ are as defined in claim 1, with H₂ in the presence of a catalyst.

4. A process for the preparation of the compound of formula (I): 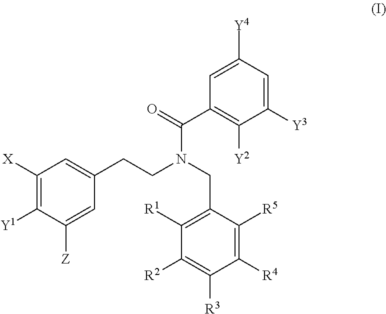
wherein X, Z, Y¹, R¹, R², R³, R⁴ and R⁵ are as defined in claim 2, which comprises
a) reacting a compound of formula (IV) 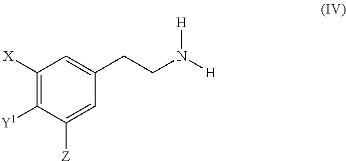
wherein X, Z and Y¹ are as defined in claim 2 with a compound of formula (V) 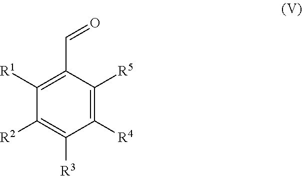
wherein R¹, R², R³, R⁴ and R⁵ are as defined in claim 2 to obtain an imine derivative of formula (VI), 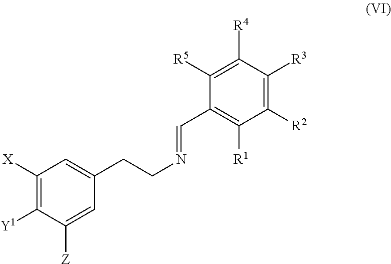
wherein X, Z, Y¹, R¹, R², R³, R⁴ and R⁵ are as defined in claim 2;
b) reacting the said imine derivative with H₂ in the presence of a metal catalyst or a reducing agent to obtain a compound of formula (II) 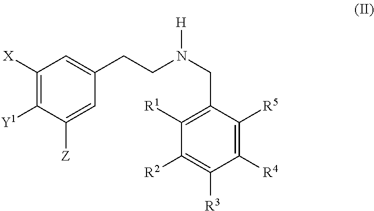
wherein X, Z, Y¹, R¹, R², R³, R⁴ and R⁵ are as defined in claim 2, with the proviso that when LiAlH₄ or Pd/C is used, none of the groups X, Z, Y¹, R¹, R², R³, R⁴ and R⁵ are chloride;
c) reacting said compound of formula (II) with a compound of formula (III): 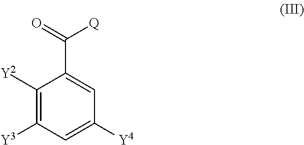
wherein Y², Y³ and Y⁴ are as defined above and Q is hydroxy or chloride in the presence of a tertiary amine with the proviso that when Q is hydroxyl, a coupling agent is present.

5. The process of claim 2, wherein the compound of formula (IV) is prepared by the reaction of compound of formula (VII) 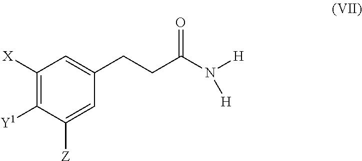
wherein X, Y¹ and Z are as defined in claim 2
with an oxidizing agent.

6. A process according to claim 5, wherein compound of formula (VII): 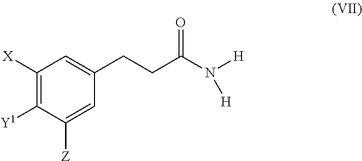
wherein X, Z and Y¹ are as defined in claim 5, is prepared by reacting compound of formula (VIII) 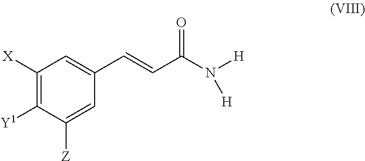
wherein X, Z and Y¹ are as defined above, with H₂ in the presence of a catalyst.

7. The process of claim 3, wherein the compound of formula (VIII) is prepared by reacting a compound of formula (IX) 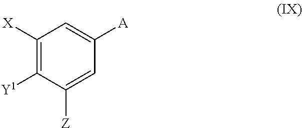
wherein X, Z and Y¹ are as defined in claim 3 and A is an halogen atom, toluenesulfonate or trifluoromethanesulfonate
with a compound of formula (X) 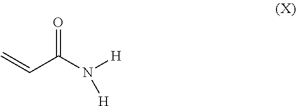
in the presence of a palladium catalyst, a combination of a palladium catalyst and a phosphine ligand or a combination of a palladium catalyst and an amine or inorganic base.

8. The process of claim 1, wherein X and Z are independently hydrogen, halogen, (C₁-C₆)alkyl, (C₁-C₃)perhaloalkyl, R¹, R², R⁴ and R⁵ are hydrogen and R³ is (C₁-C₆)alkyl.

9. The process of claim 1, wherein the compound of formula (III) wherein Q is chloride is prepared by reacting compound of formula (III) wherein Q is hydroxy with oxalyl chloride or thionyl chloride.

10. The process of claim 9, wherein for the amine coupling wherein Q is hydroxy, the coupling agent is EDC, EDC HCl, DCC, HBTU, TBTU or HOBT.

11. The process of claim 10, wherein for the amine coupling and the reductive amination, the tertiary amine is triethylamine.

12. The process of claim 9, wherein the imine derivative of formula (VI) is reacted with a metal catalyst.

13. The process of claim 12, wherein the imine derivative of formula (VI) is reacted with Pt/C, Pd/C, Ra—Ni or Ra—Co.

14. The process of claim 9, wherein the imine derivative of formula (VI) is reacted with a reducing agent.

15. The process of claim 14, wherein reducing agent is NaBH₄, Na(OAc)₃BH, LiAlH₄ or sodium bis(2-methoxyethoxy)aluminum hydride.
